(19) United States
(12) Patent Application Publication
(10) Pub. No.: US 2021/0361680 A1
(43) Pub. Date: Nov. 25, 2021

(54) INOSITOL-BASED IMMUNOTHERAPIES

(71) Applicant: NormOxys, Inc., Brighton, MA (US)

(72) Inventors: Claude NICOLAU, Newton, MA (US); Claudine KIEDA, Orleans (FR); Jean-Marie LEHN, Strasbourg (FR)

(21) Appl. No.: 17/051,668

(22) PCT Filed: May 3, 2019
(86) PCT No.: PCT/US19/30514

§ 371 Date: Oct. 29, 2020

Related U.S. Application Data
(60) Provisional application No. 62/666,151, filed on May 3, 2018.

(51) Int. Cl. A61K 31/683; A61K 39/395; A61P 35/00

(52) U.S. Cl. CPC A61K 31/683; A61P 35/00; A61K 39/3955

(57) ABSTRACT

The present invention provides, inter alia, methods and compositions that are useful in the treatment of cancer.

### PRIORITY

The present application claims priority to U.S. Provisional Application No. 62/666,151 filed May 3, 2018, the entire contents of which are hereby incorporated by reference.

### FIELD OF THE INVENTION

The present invention relates, in part, to inositol-based agent and their uses in therapy, for instance immunotherapies.

### BACKGROUND

Immunotherapies have provided great hope for cancer treatments yet their applicability appears to be limited to small responder populations. Approaches for modulating the tumor microenvironment to allow for efficient anti-tumor immune response in terms of immune cells recruitment and by deeply lowering immune checkpoints activity as by reduction of PD-L1 and PD-L2 expression are needed.

### SUMMARY OF THE INVENTION

In some aspects, the present invention relates to a method for treating, ameliorating, or preventing cancer growth, survival, metastasis, epithelial-mesenchymal transition, immunologic escape or recurrence, comprising administering an inositol-based agent and one or more immune-modulating agents, wherein the administration is simultaneous or sequential or in the context of a co-formulation. For instance, in some embodiments, the present invention relates to the use of an inositol-based agent and/or immune-modulating agent to reverse immune escape mechanisms. In some embodiments, an inositol-based agent and/or immune-modulating agent stimulates a patient's immune system to attack a tumor.

In some aspects, the present invention relates to a method for treating cancer, comprising administering an effective amount of an inositol-based agent and an effective amount of one or more immune-modulating agents to a subject in need thereof.

In some aspects, the present invention relates to a method for treating cancer, comprising administering an effective amount of an inositol-based agent to a subject in need thereof, wherein the subject is undergoing cancer therapy with one or more immune-modulating agents.

In some aspects, the present invention relates to a pharmaceutical composition comprising an effective amount of an inositol-based agent and an effective amount of one or more immune-modulating agents.

In some embodiments, the inositol-based agent is ITPP (“myo-inositol tris pyrophosphate” or “inositol-tripyrophosphate” or “inositol hexaphosphate trispyrophosphate” or “IHP-tripyrophosphate” or “OXY111A”).

In some embodiments, the immune-modulating agent is a co-stimulatory or co-inhibitory molecule. In some embodiments, the immune-modulating agent is an immune checkpoint inhibitor (CPI) and/or an immune checkpoint activator (CPA). In some embodiments, the immune-modulating agent is an agent targeting one or more of a T-cell co-stimulatory or co-inhibitory molecule, a member of the B7 family, a member of the TNF receptor or TNF ligand superfamily, a member of the TIM family, and a member of the Galectin family. In various embodiments, immune-modulating agent is an agent targeting one or more of PD-1, PD-L1, PD-L2, CD137 (4-1BB), CD137 ligand (4-1BB ligand), CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4. In various embodiments, immune-modulating agent is an agent targeting a VEGF receptor, including but not limited to VEGFR 1, VEGFR 2, and VEGFR 3.

In various embodiments, the immune-modulating agent is an antibody, including a monoclonal antibody, as well as other antibody formats.

In some embodiments, the inositol-based agent, e.g. ITPP, is combined with an immune-modulating agent that blocks, reduces and/or inhibits PD-1 and PD-L1 or PD-L2 and/or the binding of PD-1 with PD-L1 or PD-L2 (by way of non-limiting example, one or more of nivolumab, (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), MPDL328OA (ROCHE)).

In some embodiments, the immune-modulating agent is embrolizumab, nivolumab, cemiplimab, atezolizumab, avelumab, and durvalumab.

In some embodiments, the inositol-based agent, e.g. ITPP, is combined with an immune-modulating agent that increases and/or stimulates CD137 (4-1BB) and/or the binding of CD137 (4-1BB) with one or more of 4-1BB ligand and TRAF2 (by way of non-limiting example, urelumab (BMS-663513 and anti-4-1BB antibody)).

In some embodiments, the inositol-based agent, e.g. ITPP, is combined with an immune-modulating agent that blocks, reduces and/or inhibits the activity of CTLA-4, AP2M1, CD80, CD86, SHP-2, and/or PPP2R5A, and/or the binding of CTLA-4 with one or more of AP2M1, CD80, CD86, SHP-2, and PPP2R5A.

In some embodiments, the cancer treated by the present invention is pancreatic cancer. In some embodiments, the cancer treated by the present invention is liver cancer.

### BRIEF DESCRIPTION OF THE DRAWINGS

FIG. 1: Evolution of melanoma and mammary carcinoma tumor growth upon treatment by ITPP. FIG. 1, Panel a shows C57 BL6 mice received 10⁴ B16F10-Luc cells and were treated by ITPP to reach vessel normalization until day 21 and measured on day 23. FIG. 1, Panel b shows Examples of the biggest and smallest tumors obtained for B16F10 Luc cells in C57BL6 mice, in the absence of treatment (upper panel) and upon serial treatments by ITPP (lower). FIG. 1, Panel c shows Examples of the biggest and smallest tumors obtained for B16F10 Luc cells in NRj:NMRI-nude mice, in the absence of treatment (upper panel) and upon serial treatments by ITPP (lower). FIG. 1, Panel d shows BALB/c-by mice received 10⁵ 4T1 cells and were treated by ITPP to reach vessel normalization until day 21 and measured at day 31.

FIG. 2: Influence of ITPP treatment of tumors on NK cells recruitment and activation. B16 F10 Luc tumors extracted on day 23 were labelled for CD49b+, CD31+ cells detection (FIG. 2, panels a, b) and for Luc+ expressing cells (panels c, d) nuclei are detected by DAPI. FIG. 2, Panel e shows flow cytometry quantification of the immune CD45+ cells before and after treatment by ITPP. FIG. 2, Panel f shows CD45+CD49+NK cells quantification in B16F10Luc tumors upon ITPP treatment. FIG. 2, Panel g shows the effect of ITPP treatment on NK cells recruitment in B16F10Luc tumor growing in NRj:NMRI nude mice compared to normal immunocompetent C57B16 mice. FIG. 2, Panel h shows activated CD45+CD49+CD226+NK cells quantification in B16F10Luc tumor before and after ITPP treatment. FIG. 2, Panel i shows that in 4T1 mammary carcinoma, activated CD45+CD49+CD226+NK cells detected by flow cytometry before and after ITPP treatment.

FIG. 3: ITPP induced reduction of immunosuppressive myeloid derived and macrophage cell populations inside the tumor CD45+cell population. FIG. 3, panels a, b show ITPP treatment reduced the proportions of CD45+CD11b+Gr1+MDSCs in B16F10 Luc melanoma (panel a) and tended to increase CD45+ CD11c+ CD206− M1 macrophages (panel b). FIG. 3, panels c, d show ITPP treatment decreased CD45+ CD11c+ CD206+ M2 macrophages in B16F10Luc melanoma (panel c) and in 4T1 mammary carcinoma (panel d). N=6, n>2.

FIG. 4: ITPP induced reduction of inflammation mediators Th2 cell and immunosuppression mediators Treg cell populations inside the tumor CD45+cell population. FIG. 4, panel a shows ITPP treatment reduced the proportions of CD45+ CD4+ CCR4+ Th2 cells in B16F10 Luc melanoma. FIG. 4, panels b, c show ITPP treatment reduced the proportions of CD45+ CD25+ FoxP3+ cells in B16F10 Luc melanoma (panel b) and in 4T1 mammary carcinoma (panel c). N=6, n>2.

FIG. 5: Modulation of the immune checkpoint molecules PD-L1 and PD-L2 in the tumor and immune cells upon ITPP induction of tumor vessel normalization (FIG. 5, panels a, b). Dilacerated tumor cells were labelled by anti-PD-L1 and anti PD-L2 and expression analyzed by flow cytometry for total population and gated CD45− non immune cells (FIG. 5, panels c, d) and immune CD45+ cell (FIG. 5, panels e, f) populations. N=5, n>2.

FIG. 6: Influence of ITPP treatment on immune checkpoints on distinct cell types in the tumor. FIG. 6, panel a-c shows identification of PD-L1 and PD-L2 on endothelial cells from the tumor and modulation by ITPP treatment. Endothelial cells were identified on the basis of their expression of CD31 by flow cytometry are increased upon ITPP treatment (FIG. 6, panel a). CD31+ endothelial cells express less PD-L1 (FIG. 6, panel b) and less PD-L2 (FIG. 6, panel c) after ITPP treatment. FIG. 6, panel d-e shows The CD45+ immune cell population in the tumor identified by flow cytometry was enriched in PD-1 expressing cells upon ITPP treatment in B16F10 melanoma (FIG. 6, panel d) and in 4T1 mammary cacinoma (FIG. 6, panel e). FIG. 6, panel f shows flow cytometry detection of the CD47 level expression in the B16F10 tumors by flow cytometry. N=6, n>3.

FIG. 7: Chemokine receptors modulation of expression on the tumor cells, the immune and endothelial enriched cells by ITPP treatment-induced vessel normalization. FIG. 7, panel a shows flow cytometry detection of the expression of chemokines receptors on B16F10 cells in the tumor site FIG. 7, panel b shows qPCR quantification of the mRNA for chemokines and receptors expression in hypoxia as compared to normoxia. FIG. 7, panel c shows flow cytometry detection of the expression of chemokines receptors on CD45+ immune cells in the tumor site. FIG. 7, panel d shows flow cytometry detection of the expression of chemokines receptors on endothelial enriched cell population in the tumor site.

### DETAILED DESCRIPTION OF THE INVENTION

In some aspects, the present invention relates to a method for treating cancer, comprising administering an effective amount of an inositol-based agent and an effective amount of one or more immune-modulating agents to a subject in need thereof.

In some aspects, the present invention relates to a method for treating cancer, comprising administering an effective amount of an inositol-based agent to a subject in need thereof, wherein the subject is undergoing cancer therapy with one or more immune-modulating agents.

In some aspects, the present invention relates to a pharmaceutical composition comprising an effective amount of an inositol-based agent and an effective amount of one or more immune-modulating agents.

In various embodiments, the inositol-based agent and immune-modulating agent interact or produce combined effect synergistically. In various embodiments, the inositol-based agent and immune-modulating agent interact or produce combined additive effect in spite of an expected diminished effect. In various embodiments, the inositol-based agent and immune-modulating agent interact or produce combined effect that permits a reduction of dose and/or timing of treatment, and optionally a reduction of side effects, of one or more of the inositol-based agent and immune-modulating agent. Accordingly, in some embodiments, the combination of inositol-based agent and immune-modulating agent increase the therapeutic window of one or more of the inositol-based agent and immune-modulating agent.

Inositol-Based Agents

In some embodiments, the inositol-based agent of the present invention is one or more agents as described in U.S. Pat. No. 8,178,514, US Patent Publication Nos. 2008/0200437 and 2014/0142052, and International Patent Publication No. WO 2012/045009, the contents of which are hereby incorporated by reference.

In some embodiments, the inositol-based agent of the present invention is ITPP. ITPP refers to an inositol hexaphosphate with three internal pyrophosphate rings, as described in, for example, U.S. Pat. No. 8,178,514, the contents of which are hereby incorporated by reference in their entirety. In various embodiments, acids and salts of ITPP (and/or other inositol-based agents) are used. In some embodiments, ITPP (and/or other inositol-based agent) is an anion. The counterpart species to ITPP may be a counterion and the combination of ITPP with a counterion is an acid or salt. Counter ions of ITPP (and/or other inositol-based agents) may include, but are not limited to, cationic hydrogen species including protons; monovalent inorganic cations including lithium, sodium, and potassium; divalent inorganic cations including magnesium, calcium, manganese, zinc, copper and iron; polyvalent inorganic cations including iron; quaternary nitrogen species including ammonium, cycloheptyl ammonium, cyclooctyl ammonium, N,N-dimethylcyclohexyl ammonium, and other organic ammonium cations; sulfonium species including triethylsulfonium and other organic sulfonium agents; organic cations including pyridinium, piperidinium, piperazinium, quinuclidinium, pyrrolium, tripiperazinium, and other organic cations; polymeric cations including oligomers, polymers, peptides, proteins, positively charged ionomers, and other macromolecular species that possess sulfonium, quaternary nitrogen and/or charged organometallic species in pendant groups, chain ends, and/or the backbone of the polymer. An illustrative salt of an inositol-based agent, e.g. ITPP, is a monocalcium tetrasodium salt, e.g. the monocalcium tetrasodium salt of ITPP, or a mixture of sodium inositol-based agent and calcium inositol-based agent that contains about 15-25 mol % (e.g. about 15, or about 20, or about 25 mol %) calcium and about 75-85 mol % (e.g. about 75, or about 80, or about 85 mol %) sodium, e.g. a mixture of sodium ITPP and calcium ITPP that contains about 15-25 mol % (e.g. about 15, or about 20, or about 25 mol %) calcium and about 75-85 mol % sodium (e.g. about 75, or about 80, or about 85 mol %).

The invention is not limited to pairings that are purely ionic; indeed, it is well-known in the art that paired ions may evidence some degree of covalent or coordinate bond characteristic between the two components of the pair. The ITPP (and/or other inositol-based agent) acids and salts of the invention compositions may comprise a single type of counterion or may contain mixed counterions, and may optionally contain a mixture of anions of which ITPP (and/or other inositol-based agent) is one. The compositions may optionally include crown ethers, cryptands, and other species capable of chelating or otherwise complexing the counterions. The compositions may likewise optionally include acidic macrocycles or other species that are capable of complexing the ITPP (and/or other inositol-based agent) through hydrogen bonds or other molecular attractions.

ITPP (and/or other inositol-based agents), in various embodiments, may be present in various isomers. In some embodiments, ITPP is myo-inositol tris pyrophosphate or is myo-inositol (cis-1,2,3,5-trans-4,6-cyclohexanehexyl), while the invention also provides for any inositol isomer in the ITPP and/or other inositol-based agents (e.g. tripyrophosphates of the naturally occurring scyllo-, chiro-, muco-, and neo-inositol isomers, as well as those of the allo, epi-, and cis-inositol isomers).Methods of making acids and salts of ITPP are described in U.S. Pat. No. 7,084,115, the entire contents of which is incorporated herein by reference. An inositol-based agent, in some embodiments, may be made using these methods. Also, an inositol-based agent, e.g. ITPP, may be formed in vivo from a prodrug, such as by enzymatic cleavage of an ester (such as an alkyl ester) or by displacement of a leaving group such as a tolylsulfonyl group.

Also provided are methods making a pharmaceutical composition of ITPP (and/or other inositol-based agents) by mixing a sodium and calcium salt of ITPP (and/or other inositol-based agents) and a pharmaceutically acceptable adjuvant, diluent, carrier, or excipient thereof. In some embodiments, the mixture of the sodium and calcium salt of an inositol-based agent, e.g. ITPP, is obtained by mixing myo inositol tripyrophosphate-sodium salt with CaCl₂.

In some embodiments, the inositol-based agent comprises a compound represented by structure: nC⁺Aⁿ⁻, wherein: C⁺ represents independently for each occurrence an alkali metal cation (e.g., a sodium ion, a lithium ion, a potassium ion, etc.), an alkaline earth cation (e.g. a magnesium ion or calcium ion), or an ammonium cation; A represents an anionic moiety (e.g. phosphorylated inositol; IHP, wherein two phosphate groups of the IHP form an internal pyrophosphate ring; IHP, wherein 4 phosphate groups of said IHP form two internal pyrophosphate rings; IHP, wherein 6 phosphate groups of said IHP form three internal pyrophosphate rings); and n is an integer in the range of 1 to 10 inclusive (e.g. 1, or 2, or 3, or 4, or 5, or 6, or 7, or 8, or 9, or 10). In various embodiments, C⁺ is a sodium ion and Aⁿ⁻ is a phosphorylated inositol; or C⁺ is a sodium ion and Aⁿ⁻ is a phosphorylated inositol, wherein the phosphorylated inositol has one internal pyrophosphate ring; or C⁺ is a sodium ion and Aⁿ⁻ is a phosphorylated inositol, wherein the phosphorylated inositol has two internal pyrophosphate rings; or C⁺ is a sodium ion and Aⁿ⁻ s a phosphorylated inositol, wherein the phosphorylated inositol has three internal pyrophosphate rings; or C⁺ is a sodium ion and Aⁿ⁻ is IHP; or C⁺ is a sodium ion and Aⁿ⁻ is IHP, wherein two phosphate groups of said IHP form an internal pyrophosphate ring; or C⁺ is a sodium ion and Aⁿ⁻ is IHP, wherein 4 phosphate groups of said IHP form two internal pyrophosphate rings; or C⁺ is a sodium ion and Aⁿ⁻ is IHP, wherein the 6 phosphate groups of said IHP form three internal pyrophosphate rings.

In still other embodiments, inositol-based agent is one or more of those described in U.S. Pat. No. 8,178,514, US Patent Publication Nos. 2008/0200437 and 2014/0142052, and International Patent Publication No. WO 2012/045009, the contents of which are hereby incorporated by reference. For instance, in some embodiments, inositol-based agents are based on ITPP, which is altered to have one or more of: a derivatized phosphate group hydroxyl (e.g. selected from alkoxy (−OR) or acyloxy (−OCOR), where R is selected from alkyl, aryl, acyl, aralkyl, alkenyl, alkynyl, heterocyclyl, carbocycle, amino, acylamino, amido, alkylthio, sulfonate, alkoxyl, sulfonyl, or sulfoxide, or a salt derivative); the inositol in various conformations (such as, for example, cis-inositol, epi-inositol, allo-inositol, muco-inositol, neo-inositol, scyllo-inositol, (+) chiro-inositol, or (−) chiro-inositol); a substitution of inositol for another moiety (e.g. a compound that is a polyphosphate or pyrophosphate derivative of a mono-, di- or oligosaccharide containing a pyranose or furanose unit (e.g. glucose, mannose, or galactose, sucrose or lactose); or a pharmaceutical acceptable salt, stereoisomer, anomer, solvate, and hydrate thereof.

In some embodiments, the inositol-based agent is 1,6:3,4-Bis-[O-(2,3-dimethoxybutane-2,3-diyl)]-2,5-di-O-methyl-myo-inositol; 2,5-Di-O-methyl-myo-inositol; Octabenzyl 1,3,4,6-(2,5-di-O-methyl-myo-inosityl) tetrakisphosphate; Tetrasodium 1,3,4,6-(2,5-di-O-methyl-myo-inosityl) tetrakisphosphate; 1,6:3,4-Bis[O-(2,3-dimethoxybutane-2,3-diyl)]-2,5-di-O-ethyl-myo-inositol; 2,5-Di-O-ethyl-myo-inositol; Octabenzyl 1,3,4,6-(2,5-di-O-ethyl-myo-inosityl) tetrakisphosphate; Tetrasodium 1,3,4,6-(2,5-di-O-ethyl-myo-inosityl) tetrakisphosphate; 1,6:3,4-Bis[O-(2,3-dimethoxybutane-2,3-diyl)]-2,5-di-O-butyl-myo-inositol; 2,5-Di-O-butyl-myo-inositol; Octabenzyl 1,3,4,6-(2,5-di-O-butyl-myo-inosityl) tetrakisphosphate; Tetrasodium 1,3,4,6-(2,5-di-O-butyl-myo-inosityl) tetrakisphosphate; 2,5-Di-O-benzyl-1,6:3,4-bis-[O-(2,3-dimethoxybutane-2,3-diyl)]-myo-inositol; 2,5-Di-O-benzyl-myo-inositol; Octabenzyl 1,3,4,6-(2,5-di-O-benzyl-myo-inosityl) tetrakisphosphate; Tetrasodium 1,3,4,6-myo-inosityl tetrakisphosphate; Hexabenzyl 1,3,5-(2,4,6-tri-O-butyryl-myo-inosityl) trisphosphate; Hexasodium 1,3,5-(2,4,6-tri-O-butyryl-myo-inosityl) trisphosphate; Orthoformate of myo-inositol 2,4,6-tris(dibenzyl phosphate); Orthoformate of hexasodium myo-inositol 2,4,6-trisphosphate; scyllo-inositol hexakis(dibenzyl phosphate); Hexatriethylammonium scyllo-inositol hexakisphosphate; Hexatriethylammonium scyllo-inositol 1,2:3,4:5,6-trispyrophosphate; or Hexasodium scyllo-inositol 1,2:3,4:5,6-trispyrophosphate.

In some embodiments, the inositol-based agent is 1-O-methyl-α-glucose 2,3,4-trisphosphate, 1-O-methyl-α-mannose 2,3,4-trisphosphate, α-glucose 1,2,3,4-tetrakisphosphate, β-glucose 1,2,3,4-tetrakisphosphate, α-mannose 1,2,3,4-tetrakisphosphate, β-mannose 1,2,3,4-tetrakisphosphate, α-galactose 1,2,3,4-tetrakisphosphate, β-galactose 1,2,3,4-tetrakisphosphate, 1-O-methyl-α-glucose tetrakisphosphate, 1-O-methyl-α-mannose tetrakisphosphate, α-glucose pentakisphosphate, α-mannose pentakisphosphate, α-galactose pentakisphosphate, lactose octakisphosphate, sucrose octakisphosphate, or 1-O-methyl-α-glucose bispyrophosphate).

In some embodiments, the inositol-based agent is selected from diethyl-2,3-bisphospho-L-tartrate tetrasodium and di sodium salt; dibutyl-2,3-bisphospho-L-tartrate tetrasodium salt and dibutyl-cyclo-2,3-bisphospho-L-tartrate disodium salt; 2,3-bisphospho-L-tartrate hexasodium salt; tetrasodium dimethyl-meso-galactarate-2,3,4,5-tetrakisphosphate and its bispyrophosphates; tetrasodium meso-erythritol-1,2,3,4-tetrakisphosphate and its bispyrophosphates; tetrasodium pentaerythritol-2,3,4,5-tetrakisphosphate and its bispyrophosphate; tetrasodium 2,5-anhydro-D-mannitol-1,3,4,6-tetrakisphosphate and its bispyrophosphates; Diethyl-2,3-bis(dibenzylphospho)-L-tartrate; Diethyl-2,3-bisphospho-L-tartrate tetrasodium salt; Diethyl-2,3-bisphospho-L-tartrate disodium salt; Dibutyl-2,3-bis(dibenzylphospho)-L-tartrate; Dibutyl-2,3-bisphospho-L-tartrate tetrasodium salt; Dibutyl-2,3-bisphospho-L-tartrate ditriethylammonium salt; Dibutyl-cyclo-2,3-bisphospho-L-tartrate ditriethylammonium salt; Dibutyl-cyclo-2,3-bisphospho-L-tartrate disodium salt; Dibenzyl-2,3-bis(dibenzylphospho)-L-tartrate; 2,3-Bisphospho-L-tartrate hexasodium salt; Dimethyl-2,3,4,5-tetrakis(dibenzylphospho)-meso-galactarate; Tetrasodium dimethyl-meso-galactarate-2,3,4,5-tetrakisphosphate; Tetrasodium dimethyl-meso-galactarate bispyrophosphates; 1,2,3,4-Tetrakis(dibenzylphospho)-meso-erythritol; 1,2,3,4-Tetrakisphospho-meso-erythritol tetrasodium salt; Tetrasodium meso-erythritol bispyrophosphate; 1,3,4,5-Tetrakis(dibenzylphospho) pentaerythritol; Tetrasodium pentaerythritol 1,3,4,5-tetrakisphosphate; Tetrasodium pentaerythritol (1,3):(4,5)-bispyrophosphate; 1,3,4,6-Tetrakis(dibenzylphospho) 2,5-anhydro-D-mannitol; Tetrasodium 2,5-anhydro-D-mannitol 1,3,4,6-tetrakisphosphate; and Tetrasodium 2,5-anhydro-D-mannitol bispyrophosphate.

### Immune-Modulating Agents

As described herein, the inositol-based agent may be combined with one or more immune-modulating agents. In some embodiments, the immune-modulating agent is a co-stimulatory or co-inhibitory molecule (e.g. of one or more immune cells, such as, by way of non-limitation, T cells and NK cells). In some embodiments, the immune-modulating agent is an immune checkpoint inhibitor (CPI) and/or an immune checkpoint activator (CPA). See, e.g. Nature Reviews Cancer 12: 252-264 (2012), the contents of which are hereby incorporated by reference in their entirety. In some embodiments, the immune-modulating agent targets one or more biomarkers described in Semenza Cell 2012 148(3):399-408), the contents of which are hereby incorporated by reference in their entirety.

In some embodiments, the immune-modulating agent is an agent targeting one or more of a T-cell co-stimulatory or co-inhibitory molecule, an NK cell co-stimulatory or co-inhibitory molecule, a member of the B7 family, a member of the TNF receptor or TNF ligand superfamily, a member of the TIM family, and a member of the Galectin family. Accordingly, in some embodiments, the inositol-based agent, including ITPP, may be combined with an agent targeting one or more of a T-cell co-stimulatory or co-inhibitory molecule, a member of the B7 family, a member of the TNF receptor or TNF ligand superfamily, a member of the TIM family, and a member of the Galectin family.

In various embodiments, the immune-modulating agent is an agent targeting one or more of PD-1, PD-L1, PD-L2, CD137 (4-1BB), CD137 ligand (4-1BB ligand), CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4. Accordingly, in some embodiments, the inositol-based agent, including ITPP, may be combined with an agent targeting one or more of PD-1, PD-L1, PD-L2, CD137 (4-1BB), CD137 ligand (4-1BB ligand), CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4. In various embodiments, immune-modulating agent is an agent targeting a VEGF receptor, including but not limited to VEGFR 1, VEGFR 2, and VEGFR 3.

In various embodiments, the immune-modulating agent blocks, reduces and/or inhibits the binding of one or more of PD-1, PD-L1, PD-L2, 4-1BB, 4-1BB ligand, CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4 with its binding partner(s).

In some embodiments, the immune-modulating agent modulates a ligand-receptor interaction that is co-stimulatory. For example, in some embodiments, the immune-modulating agent modulates one or more of the following illustrative ligand-receptor interactions: CD80 or CD86 and CD28; B7RP1 and ICOS; CD137L and CD137; and OX040L and 0X40; CD137 (4-1BB) and CD137 ligand (4-1BB ligand); and CD70 and CD27. In some embodiments, the immune-modulating agent modulates the ligand-receptor interactions between CTLA-4 and one or more of AP2M1, CD80, CD86, SHP-2, and PPP2R5A.

In some embodiments, the immune-modulating agent modulates a ligand-receptor interaction that is inhibitory. For example, in some embodiments, the immune-modulating agent modulates one or more of the following illustrative ligand-receptor interactions: PDL-1 and/or PDL-2 and PD-1; CD80 or CD86 and CTLA-4; B7-H3 and its receptor; B7-H4 and its receptor; HVEM and BTLA; Gal9 and TIM3 and adenosine and A2aR.

In some embodiments, the immune-modulating agent is an agent that modulates myeloid-derived suppressor cells (MDSCs), including by way of non-limitation, Gr1, CD11b, Ly6C, and Ly6G. In some embodiments, the inositol-derived agent and/or immune-modulating agent reduces or eliminates one or more of expression of arginase I, production of reactive oxygen species (ROS), and production of nitric oxide (NO), for instance in the context of an MDSC (including by way of non-limitation, Gr1, CD11b, Ly6C, and Ly6G).

In some embodiments, the immune-modulating agent is an agent that modulates a Treg, including by way of non-limitation, CD4, CD25, and FoxP3.

In some embodiments, the immune-modulating agent is an agent that modulates a CD4 and/or CD8 T cell, including by way of non-limitation, CD3, CD4, CD8, PD-1, PDL-1, PDL-2, CTLA-4, CD137, CD69, CD26, TIM3, and LAG3.

In some embodiments, the immune-modulating agent is an agent that modulates NK cells, including by way of non-limitation, CD3, NKp46, CD16, NKG2D, NKp44, and NKp30.

In some embodiments, the immune-modulating agent is an agent that modulates tumor stroma and endothelium biomarkers, including by way of non-limitation, CD45, PDL-1, PDL-2, PTEN, and CD31.

In some embodiments, the immune-modulating agent modulates one or more of SLAMF4, IL-2 R α, 4-1BB/TNFRSF9, IL-2 R β, ALCAM, B7-1, IL-4 R, B7-H3, BLAME/SLAMFS, CEACAM1, IL-6 R, CCR3, IL-7 Rα, CCR4, CXCRI/IL-S RA, CCR5, CCR6, IL-10R α, CCR 7, IL-I 0 R β, CCRS, IL-12 R β1, CCR9, IL-12 R β2, CD2, IL-13 R α1, IL-13, CD3, CD4, ILT2/CDS5j, ILT3/CDS5k, ILT4/CDS5d, ILT5/CDS5a, lutegrin a 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R.

In various embodiments, the immune-modulating agent is an antibody. The antibody may be polyclonal or monoclonal; intact or truncated (e.g., F(ab′)₂, Fab, Fv); bispecific or multispecific; xenogeneic, allogeneic, syngeneic, or modified forms thereof (e.g., a chimeric antibody or a humanized antibody). In an embodiment, the immune-modulating agent is a monoclonal antibody. The monoclonal antibody may be a non-human mammal-derived monoclonal antibody, a recombinant chimeric monoclonal antibody, a recombinant humanized monoclonal antibody, or a human monoclonal antibody. In certain embodiments, the antibody further comprises an Fc region of an immunoglobulin (e.g. IgA, IgG, IgE, IgD or IgM) which may interact with Fc receptors and activate an immune response leading to depletion and/or cell death of immune cells or other cells.

An antibody, in some embodiments, refers to immunoglobulin molecules comprised of four polypeptide chains, two heavy (H) chains and two light (L) chains inter-connected by disulfide bonds capable of binding one or more antigens (e.g. bi-specific or multi-specific antibodies). Each heavy chain is comprised of a heavy chain variable region (V_{H}) and a heavy chain constant region. The heavy chain constant region is comprised of three domains, CH₁, CH₂ and CH₃. Each light chain is comprised of a light chain variable region (V_{L}) and a light chain constant region. The light chain constant region is comprised of one domain, CL. The V_{H} and V_{L} regions can be further subdivided into regions of hypervariability, termed complementarity determining regions (CDRs), interspersed with regions that are more conserved, termed framework regions (FR). Each variable region (V_{H} or V_{L}) contains 3 CDRs, designated CDR1, CDR2 and CDR3. Each variable region also contains 4 framework sub-regions, designated FR1, FR2, FR3 and FR4. The term antibody includes all types of antibodies, including, for example, IgA, IgG, IgD, IgE and IgM, and their respective subclasses (isotypes), e.g., IgG-1, IgG-2, IgG-3, and IgG-4; IgA-1 and IgA-2. An antibody, in some embodiments, also refers to antibody fragments and antigen-binding fragments.

Antibodies suitable for practicing the methods described herein can be of various antibody formats, for example, monoclonal, polyclonal, bispecific, multispecific, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain at least two antigen or target binding sites against at least two targets described herein. The immunoglobulin molecules described herein can be of any type (e.g., IgG, IgE, IgM, IgD, IgA and IgY), class (e.g., IgGI, IgG2, IgG3, IgG4, IgA1 and IgA2) or subclass of immunoglobulin molecule, as is understood by one of skill in the art. In addition, antibodies (e.g. mono-specific, bi-specific, and/or multi-specific) suitable for practicing the methods of the invention described herein can be, for example, Probodies (e.g. capped or masked prodrug antibodies (e.g. Cytomix)); Diabodies; “BITEs;” TandAbs; Flexibodies; Camelid Antibodies; dAbs; Immunobodies; Triomabs; Troybodies; Pepbodies; Vaccibodies; SIgA plAntibodies; SMIPs; NARs; IgNARs; XmABs; syn-humanisation antibodies; minibodies; RabMAbs; Fcabs; mAb2 antibodies; Sympress antibodies; UniBodies; DuoBodies; or Vascular Targeting antibodies, as described in U.S. Pat. Nos. or Patent Publication Nos. U.S. Pat. No. 7,150,872, US 2007/004909, U.S. Pat. Nos. 5,837,242, 7,235,641, US 2005/089519, US 2005/079170, U.S. Pat. No. 6,838,254, US 2003/088074, US 2006/280734, US 2004/146505, U.S. Pat. Nos. 5,273,743, 6,551,592, 6,294,654, US 2004/101905, US 2004/253238, U.S. Pat. No. 6,303,341, US 2008/227958, US 2005/043519, US 2009/148438, US 2008/0181890, US 2008/095767, U.S. Pat. No. 5,837,821, WO 2009/117531, US 2005/033031, US 2009/298195, US 2009/298195, European Patent Publication EP 2152872, WO 2010/063785, US 2010/105874, U.S. Pat. No. 7,087,411 and/or US 2010/316602, each of which is herein incorporated by reference in its entirety. See also, Storz MAbs. 2011 May-June; 3(3): 310-317.

PD-1 (also known as CD279 or Programmed cell death protein 1) is a member of the B7 family of receptors. In some embodiments, PD-1 refers to the human PD-1 sequence (see, e.g., NCBI Reference Sequence: NP_005009 herein incorporated by reference in its entirety) and any naturally occurring allelic, splice variants, and processed forms thereof. See, e.g., Keir M. E. et al., 2008. Annu Rev Immunol. 26:677-704 and UniProt:Q15116 which are hereby incorporated by reference in their entirety). PD-1 binds PD-L1 (also known as CD274 or B7-H1) and PD-L2 (also known as CD273 or B7-DC), which are also members of the B7 family. In some embodiments, PD-L1 refers to human PD-L1 (see, e.g. GenBank: AF233516 herein incorporated by reference in its entirety) and any naturally occurring allelic, splice variants, and processed forms thereof. See, e.g., UniProt: Q9NZQ7 herein incorporated by reference in its entirety. In some embodiments, PD-L2 refers to human PD-L2 (e.g. NCBI Reference Sequence: NM_025239 herein incorporated by reference in its entirety) and any naturally occurring allelic, splice variants, and processed forms thereof. See, e.g., UniProt: Q9BQ51 herein incorporated by reference in its entirety. For PD-1 and/or PD-L1 and/or PDL-2 treatments of the invention see Cancer Control July 2014, Vol. 21, No. 3 herein incorporated by reference in its entirety.

In various embodiments, the immune-modulating agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHRMACYCLICS), MPDL328OA (ROCHE). In some embodiments, the immune-modulating agent is an antibody such as, by way of non-limitation, atezolizumab (TECENTRIQ) or avelumab (BAVENCIO).

In some embodiments, the inositol-based agent is combined with one or more of BMS-936559 and MED14736 for treatment of, for example, advanced solid tumors. In some embodiments, the inositol-based agent is combined with one or more MPDL3280A (optionally with vemurafenib) and MED14736 (optionally with one or more of dabrafenib and trametinib) for the treatment of melanoma. In some embodiments, the inositol-based agent is combined with one or more MPDL3280A (optionally with erlotinib) and MED14736 (optionally with tremelimumab) for the treatment of NSCLC. In some embodiments, the inositol-based agent is combined with MPDL3280A (optionally with one or more of bevacizumab and sunitinib) for the treatment of RCC. In some embodiments, the inositol-based agent is combined with MPDL3280A for the treatment of solid or hematological malignancies. In some embodiments, the inositol-based agent is combined with one or more MPDL3280A (optionally with one or more of bevacizumab, chemotherapy and cobimetinib); MED14736 (optionally with tremelimumab) and MSB0010718C for the treatment of solid tumors. In some embodiments, the inositol-based agent is combined with AMP-224 for the treatment of advanced cancer. In some embodiments, the inositol-based agent is combined with nivolumab (optionally with iliolumbar (anti-KIR)) for the treatment of advanced solid tumors. In some embodiments, the inositol-based agent is combined with nivolumab for the treatment of castration-resistant prostate cancer, melanoma, NSCLC, and RCC. In some embodiments, the inositol-based agent is combined with pembrolizumab for the treatment of colon cancer. In some embodiments, the inositol-based agent is combined with pembrolizumab for the treatment of gastric cancer, head and neck cancer, TNBC, and urothelial cancer. In some embodiments, the inositol-based agent is combined with nivolumab (optionally with ipilimumab) for the treatment of gastric cancer, pancreatic cancer, small-cell lung cancer, and TNBC. In some embodiments, the inositol-based agent is combined with nivolumab (optionally with ipilimumab) for the treatment of glioblastoma. In some embodiments, the inositol-based agent is combined with nivolumab for the treatment of hepatocellular cancer. In some embodiments, the inositol-based agent is combined with pembrolizumab for the treatment of Hodgkin lymphoma, myeloma, myelodysplastic syndrome, and non-Hodgkin lymphoma. In some embodiments, the inositol-based agent is combined with pidilizumab for the treatment of malignant gliomas. In some embodiments, the inositol-based agent is combined with one or more of nivolumab (optionally with one or more of ipilimumab, and multiple class 1 peptides and montanide ISA 51 VG; and optionally sequentially with ipilimumab) and pembrolizumab for the treatment of melanoma. In some embodiments, the inositol-based agent is combined with pembrolizumab for the treatment of melanoma and NSCLC. In some embodiments, the inositol-based agent is combined with one or more of nivolumab (optionally with one or more of gemcitabine/cisplatin, pemetrexed/cisplatin, carboplatin/paclitaxel, bevacizumab, erlotinib, and ipilimumab) and pembrolizumab for the treatment of NSCLC. In some embodiments, the inositol-based agent is combined with pidilizumab (optionally with gemcitabine) for the treatment of pancreatic cancer. In some embodiments, the inositol-based agent is combined with pidilizumab (optionally with one or more of sipuleucel-T and cyclophosphamide) for the treatment of prostate cancer. In some embodiments, the inositol-based agent is combined with one or more of nivolumab (optionally with one or more of sunitinib, pazopanib, and ipilimumab), pembrolizumab (optionally with pazopanib), and pidilizumab (optionally with dendritic cell/RCC fusion cell vaccine) for the treatment of RCC. In some embodiments, the inositol-based agent is combined with one or more of anti-LAG3 (BMS-986016) (optionally with nivolumab), nivolumab (optionally with interleukin-21), and AMP-554 for the treatment of solid tumors. In some embodiments, the inositol-based agent is combined with pembrolizumab for the treatment of solid tumors and NSCLC.

In various embodiments, the immune-modulating agent targets one or more of CD137 or CD137L. In various embodiments, the immune-modulating agent is an antibody specific for one or more of CD137 or CD137L. For instance, in some embodiments, the immune-modulating agent is an antibody such as, by way of non-limitation, urelumab (also known as BMS-663513 and anti-4-1BB antibody). In some embodiments, the inositol-based agent is combined with urelumab (optionally with one or more of nivolumab, lirilumab, and urelumab) for the treatment of solid tumors and/or B-cell non-Hodgkins lymphoma and/or head and neck cancer and/or multiple myeloma.

In various embodiments, the immune-modulating agent targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the inositol-based agent is combined with ipilimumab (optionally with bavituximab) for the treatment of one or more of melanoma, prostate cancer, and lung cancer.

In various embodiments, the immune-modulating agent targets CD20. In various embodiments, the immune-modulating agent is an antibody specific CD20. For instance, in some embodiments, the immune-modulating agent is an antibody such as, by way of non-limitation, Ofatumumab (GENMAB), obinutuzumab (GAZYVA), AME-133v (APPLIED MOLECULAR EVOLUTION), Ocrelizumab (GENENTECH), TRU-015 (TRUBION/EMERGENT), veltuzumab (IMMU-106). In some embodiments, the immune-modulating agent is an antibody such as, by way of non-limitation, rituximab, obinutuzumab, ofatumumab, ocrelizumab, ocaratuzumab, and veltuzumab. In embodiments, the antibody capable of binding CD20 is rituximab.

### Methods of Treatment and Patient Selections

In some embodiments, the present invention relates to a method for treating, ameliorating, or preventing cancer growth, survival, metastasis, epithelial-mesenchymal transition, immunologic escape or recurrence, comprising administering by administering an inositol-based agent and one or more immune-modulating agents. Also provided herein is a method of reducing cancer recurrence, comprising administering to a subject in need thereof an inositol-based agent and one or more immune-modulating agents. The method may also prevent cancer recurrence. The cancer may be an oncological disease. The cancer may be a dormant tumor, which may result from the metastasis of a cancer. The dormant tumor may also be left over from surgical removal of a tumor. The cancer recurrence may for example, be tumor regrowth, a lung metastasis, or a liver metastasis.

In various embodiments, the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In various embodiments, the cancer is a biliary tract cancer. In some embodiments, the biliary tract cancer is selected from pancreatic cancer, gallbladder cancer, bile duct cancer, and cancer of the ampulla of Vater. In various embodiments, the cancer is liver cancer. In various embodiments, the cancer is colon cancer. In some embodiments, the biliary tract cancer is cholangiocarcinoma and/or an adenocarcinoma.

In various embodiments, the cancer is pancreatic cancer. In various embodiments, the pancreatic cancer is an exocrine tumor. In various embodiments, the pancreatic cancer is ductal adenocarcinoma. In various embodiments, the pancreatic cancer is acinar adenocarcinoma. In various embodiments, the pancreatic cancer develops from an intraductal papillary mucinous neoplasm (IPMN). In various embodiments, the pancreatic cancer is an acinar cell carcinoma, adenosquamous carcinoma, colloid carcinoma, giant cell tumor, hepatoid carcinoma, mucinous cystic neoplasms, pancreatoblastoma, serous cystadenoma, signet ring cell carcinoma, solid and pseudopapillary tumors, squamous cell carcinoma, or undifferentiated carcinoma. In various embodiments, the pancreatic cancer is an endocrine tumor or a pancreatic neuroendocrine tumors (PNETs) or islet cell tumors. In various embodiments, the pancreatic cancer is an insulinoma, glucagonoma, gastrinoma, somatostatinoma, VIPomas, and Ppomas.

In various embodiments, the pancreatic cancer is resectable, borderline resectable, locally advanced, or metastatic.

In various embodiments, the pancreatic cancer is staged using the TNM staging system. In various embodiments, the pancreatic cancer is stages I, or II, or II, or IV. In various embodiments, the pancreatic cancer is Tis, or T1, or T2, or T3, or T4. In various embodiments, the pancreatic cancer is N1 or N2. In various embodiments, the pancreatic cancer is M1.

In various embodiments, the pancreatic cancer is Stage 0, i.e. cancer in situ, in which the cancer has not yet grown outside the duct in which it started (Tis, N0, M0).

In various embodiments, the pancreatic cancer is Stage IA, i.e. the tumor is 2 cm or smaller in the pancreas. It has not spread to lymph nodes or other parts of the body (T1, N0, M0).

In various embodiments, the pancreatic cancer is Stage IB, i.e. a tumor larger than 2 cm is in the pancreas. It has not spread to lymph nodes or other parts of the body (T2, N0, M0).

In various embodiments, the pancreatic cancer is Stage IIA, i.e. the tumor is larger than 4 cm and extends beyond the pancreas. It has not spread to nearby arteries, veins, lymph nodes, or other parts of the body (T3, N0, M0).

In various embodiments, the pancreatic cancer is Stage IIB, i.e. a tumor of any size has not spread to nearby arteries or veins. It has spread to 1 to 3 regional lymph nodes but not to other parts of the body (T1, T2, or T3; N1; M0).

In various embodiments, the pancreatic cancer is Stage III. In various embodiments, the pancreatic cancer is a tumor of any size that has spread to 4 or more regional lymph nodes but not to nearby arteries, veins, or other parts of the body (T1, T2, or T3, N2, M0). In various embodiments, the pancreatic cancer is a tumor that has spread to nearby arteries and veins and may have spread to regional lymph nodes. It has not spread to other parts of the body (T4, any N, M0).

In various embodiments, the pancreatic cancer is Stage IV, i.e. any tumor that has spread to other parts of the body (any T, any N, M1).

In various embodiments, the pancreatic cancer is recurrent.

In various embodiments, the cancer is liver cancer. In various embodiments, the liver cancer described herein is primary liver cancer. In various embodiments, the primary liver cancer is one of hepatocellular carcinoma (HCC), cholangiocarcinoma, angiosarcoma, and hepatoblastoma. In some embodiments, an inositol-based agent is used in the manufacture of a medicament or in the treatment of a primary liver cancer in patient with cirrhosis. In various embodiments, the present invention includes treatment of primary liver cancers that are related to one or more of the following risk factors of liver cancer: cirrhosis, high alcohol consumption (including alcoholism), non-alcoholic fatty liver disease, infection with hepatitis viruses, smoking, low immunity, family history, diabetes, gallbladder removal, radiation from X-rays or CT scans, high body weight, betel quid consumption, and aflatoxin consumption.

In some embodiments, the liver cancer is a secondary liver cancer. In various embodiments, the secondary liver cancer is derived from one or more of the types of primary cancers that often metastasize to the liver, including, for example, colon, lung, stomach, pancreatic, breast cancers, biliary tract, esophageal, ovarian, prostate, kidney cancer, and melanoma. In some embodiments, the invention relates to a method of treating cancer, comprising administering an effective amount of an inositol-based agent neoadjuvant therapy to a patient afflicted with a tumor likely to metastasize to the liver as enumerated above. In other embodiments, the secondary liver cancer is derived from one or more of a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In various embodiments, the liver cancer described herein has one or more of a liver cancer tissue marker, selected from, for example: GPC3; GPC3+heat shock protein 70+glutamine synthetase; Telomerase; Proliferating cell nuclear antigen labeling Index; Ki-67; MIB-1 E-cadherin, and β-catenin. In various embodiments, the liver cancer described herein has one or more of a liver cancer serum marker selected from, for example: AFP; AFP-concanavalin A; AFP-LCA binding; HOC-specific AFP band on isoelectric focusing (monosialylated AFP); AFP lectin-affinity subgroups (LCAreactive LCA-L3; erythroagglutinatingphytohemagglutinin- E4 reactive AFP-P4 and P5); Circulating free AFP-IgM complexes; DCP/prothrombin produced by vitamin K absence or antagonism II; Soluble NH2 fragment of GPC-3, a heparin sulfate proteoglycan; Golgi protein 73; Iso-γGTP; Ferritin; Variant alkaline phosphatase; α1-Antitrypsin; α1-Acid glycoprotein; Osteopontin; Aldolase A; 5[prime]-Nucleotide phosphodiesterase; CK18, CK19, TPA, TPS; Circulating free squamous cell carcinoma antigen-IgM complexes; α-Fucosyl-transferase; α-L-fucosidase; Transforming growthfactor β1; Intercellular cell adhesion molecule 1; Anti-p53 antibody; Interleukin 8; Interleukin 6; Insulin-like growth factor II; Telomerase or telomerase reverse transcriptase mRNA; Vascular endothelial growth factor; Variant wild-type estrogen receptor; Vitamin B12-bindingprotein; Neurotensin; Free nucleic acids; Circulating cell-free serum DNA; Epigenetic abnormalities such as, for example, p16 hypermethylation; and Plasma proteasome.

In various embodiments, the liver cancer described herein has one or more of a liver cancer tumor cell marker selected from, for example: circulating tumor cells in peripheral blood detected by RTPCR of AFP mRNA. In various embodiments, the liver cancer described herein has one or more of a liver cancer genetic marker, selected from, for example: plasma glutamate carboxy-peptidase phospholipases A2 G13 and G7 and other cDNA microarray-derived encoded proteins; Melanoma antigen gene 1, 3; synovial sarcoma on X chromosome 1, 2, 4, 5; sarcoplasmic calcium-binding protein 1; New York esophageal squamous cell carcinoma 1; and Circulating methylated DNA (ras association domain family 1A).

In some embodiments, the liver cancer expresses alpha-fetoprotein. Further details of markers that define, in some embodiments, the liver cancers of the present invention are found at, for example, Sturgeon, et al. Use of Tumor Markers in Liver, Bladder, Cervical, and Gastric Cancers American Association for Clinical Chemistry, Inc. (2010), the contents of which are hereby incorporated by reference.

In various embodiments, the liver cancer described herein is classified as one or more of localized resectable, localized unresectable, advanced and recurrent.

Localized resectable liver cancer (some T1 or T2, N0, M0 tumors) refers to an early stage cancer. Often the size of the tumor(s) is small and nearby blood vessels are not affected. Further, this type of cancer is often characterized by generally acceptable liver function and general health. In these embodiments, an inositol-based agent may be used as the sole treatment to shrink small tumors and obviate the need for surgery (especially in, for example, patients that cannot easily undergo surgery, like the elderly). Or, in these embodiments, an inositol-based agent may be used as an adjuvant or neoadjuvant therapy to compliment, for example, surgical resection and improve clinical outcome.

Localized unresectable liver cancer (some T1 to T4, N0, M0 tumors) refers to cancers that haven't yet spread, but that can't be removed safely by surgical resection for various reasons (e.g. a tumor is too large to be removed safely, a tumor is in a part of the liver that makes it hard to remove (such as very close to a large blood vessel). there are several tumors, and the rest of the liver is unhealthy (because of cirrhosis or other reasons)). These patients may be treated with a liver transplant if it is possible. In these embodiments, an inositol-based agent may be used as a therapy that bridges the treatment gap to transplant (e.g. maintains patient health and/or suppresses tumor growth and/or metastasis until transplantation is possible). In some cases, an inositol-based agent treatment may shrink the tumor(s) enough so that surgery (surgical resection or transplant) may become possible.

Advanced liver cancer (includes all N1 or M1 tumors) refers to cancers that have spread outside the liver (either to the lymph nodes or to other organs). Because these cancers are widespread, they cannot be treated with surgery. If the liver is functioning well enough (e.g. Child-Pugh class A or B), an inositol-based agent alone or as a combination therapy may help control the growth of the cancer for a time and may extend life.

Recurrent liver cancer refers to cancer that after treatment. Recurrence can be local (in or near the same place it started) or distant (spread to organs such as the lungs or bone). Treatment of liver cancer that returns after initial therapy depends on many factors, including where it comes back, the type of initial treatment, and how well the liver is functioning. Patients with localized resectable disease that recurs in the liver might be eligible for further surgery or local treatments like ablation or embolization. If the cancer is widespread, targeted therapy or chemotherapy may be options. In all of these scenarios, an inositol-based agent may be used to compliment or supplant treatment plans.

In various embodiments, the liver cancer described herein is classified with the AJCC (TNM) staging system. Stages are labeled using Roman numerals I through IV (1-4). Some stages are further sub-divided into A and B or even C. For the most part, the lower the number, the less the cancer has spread. A higher number, such as stage IV (4), means a more advanced cancer. The staging systems for most types of cancer depend only on the extent of the cancer, but most patients with liver cancer have damage to the rest of their liver along with their cancer. This means that the liver might not be working as well as it should, which also affects treatment options and the outlook for the patient. The inositol-based agent e finds uses across this spectrum of stages (e.g. IA, or IB, or IC, or IIA, or IIb, or IIIC, or IIIA, or IIIB, or IIIC, or IVA, or IVB, or IVC, including one or more of TX, T0, Tis, T1, T2, T3, T4, NX, N0, N1, N2, N3, MX, M0, M1 and any grades of 1, or 2, or 3, or 4, or 5). For example, an inositol-based agent is useful to provide liver cancer cure or attenuation in lower stages or it may be used as a palliative treatment in higher stages. In all stages, an inositol-based agent may be used as an adjuvant or neoadjuvant.

The present invention also provides methods for treating a hyper-proliferative condition comprising administering to a subject in need thereof a therapeutically effective amount of the agents described herein and/or pharmaceutical compositions described herein, wherein the hyper-proliferative condition is not cancer or characterized by undesired angiogenesis. Hyper-proliferative conditions that may be treated by the methods of the present invention include, but not limited to: diabetic nephropathy, glomerulosclerosis, IgA nephropathy, cirrhosis, biliary atresia, congestive heart failure, scleroderma, radiation-induced fibrosis, lung fibrosis (idiopathic pulmonary fibrosis, collagen vascular disease, sarcoidosis, interstitial lung diseases and extrinsic lung disorders), psoriasis, genital warts and hyperproliferative cell growth diseases, including hyperproliferative keratinocyte diseases such as hyperkeratosis, ichthyosis, keratoderma or lichen planus. In some embodiments, the tissue or organ displaying the hyperproliferative condition is hypoxic. In a further embodiment, the method for treating a hyper-proliferative condition further comprises administering an additional antihyperproliferative agent, such as those described herein.

In some embodiments, the present invention relates to treatment of a PTEN Hamartoma Tumor syndrome (PHTS), which includes several syndromes including Cowden syndrome (CS), Bannayan-Riley-Ruvalcaba syndrome (BRRS), Proteus syndrome (PS), and Autism Spectrum Disorder (ASD). In specific embodiments, the present invention relates to treatment of cancers which frequently display genetic inactivation of PTEN, including without limitation glioblastoma, endometrial cancer, and prostate cancer; and/or treatment of cancers which frequently display reduced expression of PTEN, including without limitation lung and breast cancer.

In some embodiments, the inositol-based agent and/or immune-modulating agent is used to treat a subject that has a treatment-refractory cancer. In some embodiments, the inositol-based agent is used to treat a subject that is refractory to one or more immune-modulating agents. For instance, in some embodiments, the subject is refractory to a PD-1 and/or PD-L1 and/or PDL-2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), atezolizumab (TECENTRIQ), avelumab (BAVENCIO).and/or MPDL328OA (ROCHE)-refractory patients. For instance, in some embodiments, the subject is refractory to an anti-CTLA-4 agent, e.g. ipilimumab (Yervoy)-refractory patients (e.g. melanoma patients). In some embodiments, the subject is refractory to an inositol-based agent. Accordingly, in various embodiments the present invention provides methods of cancer treatment that rescue patients that are non-responsive to various therapies, including monotherapy of an inositol-based agent or one or more immune-modulating agents. In some embodiments, the subject is characterized by PD-L1+ MDSC infiltration at the tumor site and/or TME which has immunosupressive effects.

In some embodiments the inositol-based agent and/or immune-modulating agent is used to treat cancers of various stages (e.g. Stage I, or II, or III, or IV). By way of non-limiting example, using the overall stage grouping, Stage I cancers are localized to one part of the body; Stage II cancers are locally advanced, as are Stage III cancers. Whether a cancer is designated as Stage II or Stage III can depend on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body. In some embodiments, the inositol-based agent (and/or the immune-modulating agent) reduces side effects of the therapies as a patient experiences individually. For example, the combination therapy of an inositol-based agent and one or more immune-modulating agent may allow for a lower dose of the inositol-based agent and/or one or more immune-modulating agent (e.g. as compared to monotherapy) and thereby increase the therapeutic window of either agent. In some embodiments, the lowered dose mitigates one or more side effects without loss of efficacy (or minimal loss of efficacy).

In some embodiments, the inositol-based agent causes vessel normalization.

In some embodiments, the inositol-based agent (and/or the immune-modulating agent) promotes or stimulates the activity or activation of one or more immune cells including, but not limited to, cytotoxic T lymphocytes, T helper cells, natural killer (NK) cells, natural killer T (NKT) cells, anti-tumor macrophages (e.g. M1 macrophages), and dendritic cells.

In some embodiments, optionally and without wishing to be bound by theory by causing vessel normalization, the inositol-based agent (and/or the immune-modulating agent) promotes or stimulates the activity or activation of one or more immune cells including, but not limited to, cytotoxic T lymphocytes, T helper cells, natural killer (NK) cells, natural killer T (NKT) cells, anti-tumor macrophages (e.g. M1 macrophages), and dendritic cells.

In some embodiments, the inositol-based agent (and/or the immune-modulating agent) promotes or stimulates the activity and/or activation of T cells, including, by way of a non-limiting example, activating and/or stimulation one or more signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: proinflammatory cytokine production or T cell migration or T cell tumor infiltration.

In some embodiments, the inositol-based agent (and/or the immune-modulating agent) modulates the immune system in favor of immune response to one or more tumors via a cell-medicated immune response, including the innate immune system and/or the adaptive immune system.

In some embodiments, the inositol-based agent (and/or the immune-modulating agent) inhibits or reduces immune modulation or immune tolerance to tumor cells. In some embodiments, the combination therapy of an inositol-based agent and one or more immune-modulating agents inhibits or reduces the activity or activation of one or more cells including, but not limited to: myeloid-derived suppressor cells (MDSCs), regulatory T cells (Tregs); tumor associated neutrophils (TANs), M2 macrophages, and tumor associated macrophages (TAMs). In some embodiments, the inositol-based agent (and/or the immune-modulating agent) inhibits or reduces the activity or activation of Th17 cells.

In some embodiments, the inositol-based agent (and/or the immune-modulating agent) reduces or eliminates the infiltration of immunosuppressive cells to the tumor site and/or TME. For example, in some embodiments, the present agents prevent, reduce or eliminate the infiltration of one or more of regulatory T cells (Tregs); myeloid suppressor cells; tumor associated neutrophils (TANs), M2 macrophages, and tumor associated macrophages (TAMs) to the tumor site and/or TME. In some embodiments, the present agents inhibit or reduce the activity or activation of one or more of regulatory T cells (Tregs); myeloid suppressor cells; tumor associated neutrophils (TANs), M2 macrophages, and tumor associated macrophages (TAMs).

In some embodiments, the inositol-based agent (and/or the immune-modulating agent) stimulates the immune response in concert with, for example, a co-stimulatory agent or in contrast to, for example, a co-inhibitory agent. In some embodiments, the inositol-based agent causes an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g. one or more of M1 and M2) into a tumor or the tumor microenvironment. In some embodiments, the inositol-based agent causes the infiltration, or an increase in infiltration, of CD45+ cells into a tumor or the tumor microenvironment. In some embodiments, the inositol-based agent causes the infiltration of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g. one or more of M1 and M2) into a tumor or the tumor microenvironment. In some embodiments, the inositol-based agent causes the infiltration of CD45+ cells into a tumor or the tumor microenvironment. For example, in some embodiments, the inositol-based agent modulates the CCR5/CCL5 and/or CCR10/CCL10 and/or CXCR4/CXCL12 and/or CCR7/CCL21 and/or CXCR3 and/or CCR10/CXCL28 axes. For example, in some embodiments, the inositol-based agent upregulates the expression of CCR5 and/or CCR10 and/or CXCR4, optionally in the context of a CD45+ cell. In some embodiments, the inositol-based agent causes the infiltration of CD11c+ cells (e.g. DCs, M1 macrophages) into a tumor or the tumor microenvironment. In some embodiments, the inositol-based agent causes the infiltration of CD40+ cells (e.g. APCs, such as B cells, DCs, M1 macrophages) into a tumor or the tumor microenvironment. In various embodiments, the inositol-based agent causes an upregulation of CD49b, which is found on, for example, T cells (e.g. NKT cells), NK cells, fibroblasts and platelets. In some embodiments, the inositol-based agent causes the infiltration of CD146+ cells (e.g. NK cells and neutrophils). For example, in some embodiments, the inositol-based agent modulates the CCR10/CCL10 axis, for example, by upregulating the expression of CCR10, optionally in the context of a CD146+ cell.

In some embodiments, the inositol-based agent (and/or the immune-modulating agent) causes an increase of intratumor NKs and/or an increase of NKs in the TME. In various embodiments, an increase of infiltration of NKs through the endothelial barrier is provided. In some embodiments, the inositol-based agent (and/or the immune-modulating agent) causes an increase of CD8+ T cells and/or B cells and/or CD40+ endothelial cells (ECs) in the tumor site and/or TME.

In some embodiments, the tumor microenvironment contemplated described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In some embodiments, the tumor microenvironment contemplated described herein comprises cancer-associated fibroblasts (CAFs).

In various embodiments, the present therapies provide a lasting immunotherapeutic effect. For example, the present therapies, in some embodiments, eliminate components of the microenvironment, which can reinstate a tumorigenic milieu and contribute to recurrence. For example, a reduction or elimination of CAFs, which play a role in facilitating tumor growth and metastatic dissemination, is provided. In some embodiments, the present therapies may comprise any of the CAF-reducing agents described in Immunotherapy, November 2012; 4(11): 1129-1138, the contents of which are hereby incorporated by reference. See also Tejchman, et al. Oncotarget 2017 May 9; 8(19): 31876-31887 and Suchanski, et al. 2017 PLoS ONE 12(9): e0184970. doi.org/10.1371/journal.pone.0184970, the contents of which are hereby incorporated by reference.

For example, in some embodiments, the inositol-based agent (and/or the immune-modulating agent) reduces the infiltration of CD25+ Fox-P3 cells in the tumor site. That is, in some embodiments, the inositol-based agent reduces the infiltration of cells that suppress or downregulate induction and proliferation of effector T cells.

In some embodiments, the inositol-based agent (and/or the immune-modulating agent) modulates cancer stem cell (CSC)-like and/or epithelial mesenchymal transition (EMT) phenotypes. In some embodiments, the inositol-based agent (and/or the immune-modulating agent) prevents or reduces metastasis. In some embodiments, the inositol-based agent (and/or the immune-modulating agent) targets and/or reduces and/or eliminates ABCG2+ cells as described in, for example, Life Sciences 86 (17-18) 24 Apr. 2010, Pages 631-637, the entire contents of which are hereby incorporated by reference. See also Kieda, et al. J Mol Med (2013) 91: 883. doi.org/10.1007/s00109-013-0992-6, the entire contents of which are hereby incorporated by reference.

In some embodiments, the inositol-based agent (and/or the immune-modulating agent) reduces or eliminates the effects of various pro-angiogenic molecules such as VEGF, PDGF, etc. For instance, various pro-angiogenic molecules may suppress the immune system, e.g. by attracting immunosuppressive cells to infiltrate a tumor (by way of non-limitation, M2 macrophages). In some embodiments, the inositol-based agent (and/or the immune-modulating agent) reduces or eliminates the activity and/or expression of one or more of a pro-angiogenic molecule and it cognate receptor, for instance by reducing or eliminating receptor/agonist binding (e.g. VEGF and/or VEGF receptor, see Collet et al. 2014 Mol Cancer Ther 13, 165-178, Collet, et al. 2016 Cancer Lett 370, 345-357, and Klimkiewicz, et al. 2017 Cancer Lett. Volume 396, 28 June 2017, Pages 10-20, the entire contents of which are hereby incorporated by reference, PDGF and/or PDGF receptor). In some embodiments, the inositol-based agent (and/or the immune-modulating agent) is combined with an agent that blocks VEGF and VEGF receptor and/or PDGF and PDGF receptor interactions, including, for example sunitinib (SUTENT, PFIZER), bevacizumab (AVASTIN, GENENTECH/ROCHE), ranibizumab (LUCENTIS, GENENTECH/NOVARTIS). By way of illustration, the inositol-based agent may be combined with one or more of sunitinib, an anti-CD137 antibody, and IL-12 in treatment of cancers, including without limitation colon adenocarcinoma.

In some embodiments, the inositol-based agent (and/or the immune-modulating agent) causes a reduction in the activity and/or expression of osteopontin. Kieda, et al. J Mol Med (2013) 91: 883. doi.org/10.1007/s00109-013-0992-6, the entire contents of which are hereby incorporated by reference.

In some embodiments, the inositol-based agent increases tumor pO₂. In some embodiments, the inositol-based agent causes vessel normalization and/or microenvironment modification. In some embodiments, the inositol-based agent downregulates the PD-1/PD-L1 (and/or PD-L2) interaction by down regulating the expression of one of more of PD-1, PD-L1, and PD-L2. In some embodiments, this reduction of the PD-1/PD-L1 (and/or PD-L2) interaction is bolstered by use of the inositol-based agent with an immune-modulating agent.

In various embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient or a subject. In some embodiments, the human is a female. In some embodiments, the human is a male.

### Treatment Regimens and Combination Therapies

In some embodiments, present invention provides for specific cancer treatment regimens with inositol-based agents and immune-modulating agents (and optionally one or more additional therapeutic agent). For example, in some embodiments, the inositol-based agent, e.g. ITPP, is administered to a patient first to normalize tumor vascularization, optionally by reducing or ablating hypoxia. Such first administration of the inositol-based agent, e.g. ITPP, may stimulate and/or increase T lymphocytes (e.g. CD4+ and CD8+ T cells) and/or NK cells tumor and/or TME infiltration and/or inhibit and/or decrease recruitment of immunosuppressive cells (e.g. myeloid-derived suppressor cells (MDSCs), regulatory T cells (Tregs); tumor associated neutrophils (TANs), M2 macrophages, and tumor associated macrophages (TAMs)) to the tumor and/or TME. In some embodiments, the present therapies may alter the ratio of M1 versus M2 macrophages in the tumor site and/or TME to favor M1 macrophages. Notably, unlike for example, anti-angiogenic molecules, the inositol-based agents, in some embodiments, induce a long lasting (i.e. greater than transient) vascular normalization. For example, inositol-based agent-vascular normalization may last greater than 1, or 2, or 3, or 4, or 5, or, or 6, or 7, or 14 days, or 21 days. Accordingly, in some embodiments, this long-lasting inositol-based agent-vascular normalization allows for a sustainable permissive tumor microenvironment which is more likely to be responsive to one or more immune-modulating agents. That is, in some embodiments, the inositol-based agent potentiates immune-modulating agent therapy.

Alternatively, the inositol-based agent, e.g. ITPP, is administered to a patient after treatment with one or more immune-modulating agents. For instance, in some embodiments, the immune-modulatory agent targets one or more co-inhibitory molecules and reduces or eliminates immunosupression. In this favorable context, i.e. upon removal of suppression, the inositol-based agent, e.g. ITPP, is administered is administered to stimulate the immune system. Or the immune-modulatory agent targets one or more co-stimulatory molecules first and the inositol-based agent, e.g. ITPP, is administered is administered second to bolster this effect, for example, synergistically.

Further, as described herein, the inositol-based agent and/or immune-modulating agent can be combined with an additional therapeutic agent in the context of, for example, co-administration, a treatment regimen or a co-formulation.

In some embodiments, the inositol-based agent and/or immune-modulating agent, optionally with an additional therapeutic agent, can be administered sequentially. The term “sequentially” as used herein means that the additional therapeutic agent and the inositol-based agent and/or immune-modulating agent are administered with a time separation of more than about 60 minutes. For example, the time between the sequential administration of the additional therapeutic agent and the inositol-based agent and/or immune-modulating agent can be more than about 60 minutes, more than about 2 hours, more than about 5 hours, more than about 10 hours, more than about 1 day, more than about 2 days, more than about 3 days, or more than about 1 week apart. The optimal administration times may depend on the rates of metabolism, excretion, and/or the pharmacodynamic activity of the additional therapeutic agent and the inositol-based agent and/or immune-modulating agent being administered. Either the additional therapeutic agent or the present agents may be administered first.

In some embodiments, the inositol-based agent and/or immune-modulating agent, optionally with an additional therapeutic agent, can be administered simultaneously. The term “simultaneously” as used herein, means that the additional therapeutic agent and the inositol-based agent and/or immune-modulating agent are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and inositol-based agent and/or immune-modulating agent can be by simultaneous administration of a single formulation (e.g., a formulation comprising the additional therapeutic agent and the inositol-based agent and/or immune-modulating agent) or of separate formulations (e.g., a first formulation including the additional therapeutic agent and a second formulation including the inositol-based agent and/or immune-modulating agent).

Co-administration also does not require the additional therapeutic agents to be administered to the subject by the same route of administration. Rather, each therapeutic agent can be administered by any appropriate route, for example, parenterally or non-parenterally.

Such a combination may lead to synergism and/or additive and/or potent effects at a lower dose of the inositol-based agent and/or immune-modulating agent. For example, when the inositol-based agent is combined with one or more immune-modulating agents the effective amount of the inositol-based agent may be lower than what it would be in a monotherapy. In some embodiments, the inositol-based agent is combined with an immune-modulating agent and the effective amount of the inositol-based agent is a sub-therapeutic dose. For example, when the immune-modulating agent is combined with an inositol-based agent the effective amount of the immune-modulating agent may be lower than what it would be in a monotherapy. In some embodiments, the immune-modulating agent is combined with an inositol-based agent and the effective amount of the immune-modulating agent is a sub-therapeutic dose. In various embodiments, the immune-modulating agent is combined with an inositol-based agent and an additional therapeutic agent and the effective amount of the additional therapeutic agent is a sub-therapeutic dose The term “sub-therapeutic dose or amount” means that a dose or amount of a pharmacologically active substance is below the dose or amount of that substance that is administered, as the sole substance, to achieve a therapeutic effect. The sub-therapeutic dose of such a substance may vary depending upon the subject and disease condition being treated, the weight and age of the subject, the severity of the disease condition, the manner of administration and the like, which can readily be determined by one of ordinary skill in the art. In one embodiment, the sub-therapeutic dose or amount of the chemotherapeutic agent is less than 90% of the approved full dose of the chemotherapeutic agent, such as that provided in the U.S. Food & Drug Administration-approved label information for the chemotherapeutic agent. In other embodiments, the sub-therapeutic dose or amount of the chemotherapeutic agent is less than 80%, 70%, 60%, 50%, 40%, 30%, 20% or even 10% of the approved full dose, such as from 20% to 90%, 30% to 80%, 40% to 70% or another range within the values provided herein.

In some embodiments, the effective amount of the immune-modulating agent is less than an effective amount used in monotherapy for the same cancer and/or a combination therapy with an agent besides an inositol-based agent for the same cancer. In some embodiments, the effective amount of the inositol-based agent is less than an effective amount used in monotherapy for the same cancer and/or a combination therapy with an agent besides an immune-modulating agent for the same cancer.

In various embodiments, the inositol-based agent is combined with one or more immune-modulating agents (e.g. 1, or 2, or 3, or 4, or 5 immune-modulating agents) and, optionally, one or more additional therapeutic agents (e.g. 1, or 2, or 3, or 4, or 5 additional therapeutic agents). Such combinations may lead to synergism and/or additive and/or potent effects at a lower dose of the inositol-based agent and/or immune-modulating agent and/or the one or more additional therapeutic agents. Co-administration may be simultaneous or sequential. Further the pharmaceutical compositions including the inositol-based agent and/or immune-modulating agent may comprise the additional therapeutic agent (e.g. via co-formulation). That is, in some embodiments, two or more of any of the agents disclosed herein may be co-formulated. Further, in some embodiments, the inositol-based agent and/or immune-modulating agent may be administered to a patient that is undergoing treatment with one or more additional therapeutic agent. Further, in some embodiments, the inositol-based agent and/or immune-modulating agent may supplant a patient's current treatment with one or more additional therapeutic agent.

Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the agents described herein are used as an adjuvant therapy in the treatment of a cancer. In some embodiments the therapeutic agents described herein are administered as a neoadjuvant therapy prior to resection. In certain embodiments, neoadjuvant therapy refers to therapy to shrink and/or downgrade the tumor prior to any surgery. In some embodiments, neoadjuvant therapy means a therapeutic agent described herein is administered to cancer patients prior to surgery. In some embodiments the therapeutic agents described herein are useful as a maintenance therapy after an initial treatment with a first-line therapy, including without limitation any of the additional therapeutic agents of the present disclosure.

In various embodiments, the present invention provides vessel normalization and long lasting pO₂ restoration. In various embodiments, the present invention increases the efficacy of the radiotherapy. In various embodiments, the present invention provides vessel normalization and long lasting pO₂ restoration and increases the efficacy of the radiotherapy.

In various embodiments, the present invention provides a treatment regimen or a method for treating cancer or tumors in a subject that includes administering simultaneously or sequentially a therapeutically effective amount of an inositol-based agent and/or an immune-modulating agent and one or more of the additional therapeutic agents described herein. In various embodiments, the present invention provides a treatment regimen or a method for treating cancer or tumors in a subject that includes administering simultaneously or sequentially a therapeutically effective amount of an inositol-based agent and/or an immune-modulating agent and one or more of the anti-cancer agents described herein, including but not limited to chemotherapeutic agents. Suitable chemotherapeutic agents to be used in the methods of the present invention may include those described herein. In certain embodiments, the chemotherapeutic agent is one or more of 5-fluorouracil (5-FU), doxorubicin, gemcitabine, paclitaxel, and cisplatin. By way of example, in some embodiments, the present invention provides combining an inositol-based agent and/or an immune-modulating agent with one or more common cancer treatment regimens (by way of non-limiting illustration, FOLFOX, FOLFIRI, IFL, FL (Mayo), QUASAR, Machover schedule, CAF, CMF, ECF, and FEC).

In various embodiments, the additional therapeutic agent is an anti-cancer agent, which includes, but is not limited to, a chemotherapeutic agent. In various embodiments, the anti-cancer agent is selected from, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; ionidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib; inhibitors of the enzyme Bruton's tyrosine kinase (BTK) like Ibrutinib, inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In an embodiment, the chemotherapeutic agent is a microtubule-targeting agent such as paclitaxel. In another embodiment, the chemotherapeutic agent is a DNA-intercalating agent such as platinum-based agents (e.g., cisplatin) or doxorubicin. In a further embodiment, the chemotherapeutic agent is a nucleoside metabolic inhibitor such as gemcitabine or capecitabine.

In some embodiments, the additional therapeutic agent is aminoglutethimide, amsacrine, anastrozole, asparaginase, beg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In various embodiments, the additional therapeutic agent is an antihyperproliferative agent. Antihyperproliferative agents include, but are not limited to, doxorubicin, daunorubicin, mitomycin, actinomycin D, bleomycin, cisplatin, VP16, an enedyine, taxol, vincristine, vinblastine, carmustine, melphalan, cyclophsophamide, chlorambucil, busulfan, lomustine, 5-fluorouracil, gemcitabin, BCNU, or camptothecin.

In addition, the additional therapeutic agent can further include the use of radiation. In addition, the methods of treatment can further include the use of photodynamic therapy.

### Salts, Pharmaceutical Compositions and Doses

In some embodiments, the agents described herein include derivatives that are modified, i.e., by the covalent attachment of any type of molecule to the agent such that covalent attachment does not prevent the activity of the agent. For example, but not by way of limitation, derivatives include agents that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, or formylation. Additionally, the derivatives can contain one or more non-classical amino acids.

In still other embodiments, the agents described herein may be modified to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and a targeting agent.

In yet other embodiments, the present invention provides for the agents described herein and pharmaceutically acceptable esters, prodrugs, salts, solvates, enantiomers, stereoisomers, active metabolites, co-crystals, and other physiologically functional derivatives thereof.

In an embodiment, the agent described herein is in the form of a pharmaceutically acceptable salt, namely those salts which are suitable for use in contact with the tissues of humans and other animals without undue toxicity, irritation, allergic response and the like, and are commensurate with a reasonable benefit/risk ratio. Pharmaceutically acceptable salts are well known in the art. The salts can be prepared in situ during the final isolation and purification of the agent, or separately by reacting the free base function with a suitable acid or a free acid functionality with an appropriate alkaline moiety. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In one aspect, the present invention provides agents described herein, and a pharmaceutically acceptable carrier or excipient. The pharmaceutical composition can be in any suitable form appropriate for the desired use and route of administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable pharmaceutical excipients are described in Remington's Pharmaceutical Sciences 1447-1676 (Alfonso R. Gennaro eds., 19th ed. 1995), incorporated herein by reference.

Additionally, the pharmaceutical compositions of the present invention may contain adjuvants such as preservatives, wetting agents, emulsifying agents, pH buffering agents, and dispersing agents. Further, auxiliary, stabilizing, thickening, lubricating, and coloring agents can be included. Prevention of the action of microorganisms may be ensured by the inclusion of various antibacterial and antifungal agents, for example, paraben, chlorobutanol, phenol sorbic acid, and the like. The pharmaceutical compositions may also include isotonic agents such as sugars, sodium chloride, and the like.

Where necessary, the pharmaceutical compositions can also include a solubilizing agent. Also, the agents can be delivered with a suitable vehicle or delivery device as known in the art. Compositions for administration can optionally include a local anesthetic such as, for example, lidocaine to lessen pain at the site of the injection.

The pharmaceutical compositions of the present invention can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. In one embodiment, the pharmaceutical composition is in the form of a capsule. In another embodiment, the pharmaceutical composition is in the form of a tablet.

In some embodiments, the administration of any of the described agents is any one of oral, intravenous, and parenteral. In various embodiments, routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically, for example, to the ears, nose, eyes, or skin. In some embodiments, the administering is effected orally or by parenteral injection. The mode of administration can be left to the discretion of the practitioner, and depends in-part upon the site of the medical condition. In various embodiments, administration results in the release of any agent described herein into the bloodstream.

Any agent and/or pharmaceutical composition described herein can be administered orally. Such agents and/or pharmaceutical compositions can also be administered by any other convenient route, for example, by intravenous infusion or bolus injection, by absorption through epithelial or mucocutaneous linings (e.g., oral mucosa, rectal and intestinal mucosa, etc.) and can be administered together with an additional therapeutic agent. Administration can be systemic or local. Various delivery systems are known, e.g., encapsulation in liposomes, microparticles, microcapsules, capsules, etc., and can be used. In specific embodiments, it may be desirable to administer locally to the area in need of treatment.

In one embodiment, an agent described herein and/or pharmaceutical composition described herein is formulated in accordance with routine procedures as a composition adapted for oral administration to humans. Solid dosage forms for oral administration include, for example, capsules, tablets, pills, powders, and granules. In such dosage forms, the active agent is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium agents, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Non-limiting examples of embedding compositions which can be used include polymeric substances and waxes.

The active agents can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions, and by the use of surfactants.

Any agent described herein and/or pharmaceutical composition described herein can be administered by controlled-release or sustained-release means or by delivery devices that are known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, Eudragit, other polymer matrices, gels, permeable membranes, osmotic systems, multilayer coatings, microparticles, liposomes, microspheres, or a combination thereof to provide the desired release profile in varying proportions. Suitable controlled- or sustained-release formulations can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Formulations comprising the agents described herein and/or pharmaceutical compositions of the present invention may conveniently be presented in unit dosage forms and may be prepared by any of the methods known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

It will be appreciated that the actual dose of the agents described herein and/or pharmaceutical compositions of the present invention to be administered according to the present invention may vary according to the particular agent, the particular dosage form, and the mode of administration. Many factors that may modify the action of the inositol-based agents (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the agents described herein and/or pharmaceutical compositions of the present invention can be administered in unit dosage forms (e.g., tablets or capsules) containing, for example, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg per unit dosage form. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween.

In some embodiments, the agents described herein and/or pharmaceutical compositions of the present invention are administered at an amount of from about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, or from about 0.1 mg to about 1 mg daily.

In various embodiments, the agents described herein and/or pharmaceutical compositions of the present invention are administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween.

In some embodiments, a suitable dosage of the agents described herein and/or pharmaceutical compositions of the present invention is in a range of about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween. In other embodiments, a suitable dosage of the inositol-based agent and/or immune-modulating agent and/.or additional therapeutic agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the invention, the agents and/or pharmaceutical compositions described herein may be administered, for example, more than once daily, about once per day, about every other day, about every third day, about once a week, about once every two weeks, about once every month, about once every two months, about once every three months, about once every six months, or about once every year.

### Kits

The invention also provides kits that can simplify the administration of the agents and/or pharmaceutical compositions described herein. The kit is an assemblage of materials or components, including at least one of the agents described herein. The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to affect a desired outcome, such as to treat, for example, cancer, diabetes, or obesity. Optionally, the kit also contains other useful components, such as, diluents, buffers, pharmaceutically acceptable carriers, syringes, catheters, applicators, pipetting or measuring tools, bandaging materials or other useful paraphernalia as may be readily recognized by those of skill in the art.

The materials and components assembled in the kit can be provided to the practitioner store in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

This invention is further illustrated by the following non-limiting examples.

### EXAMPLES

### Tumor Vessel Normalization by ITPP Procedure and pO2 Status

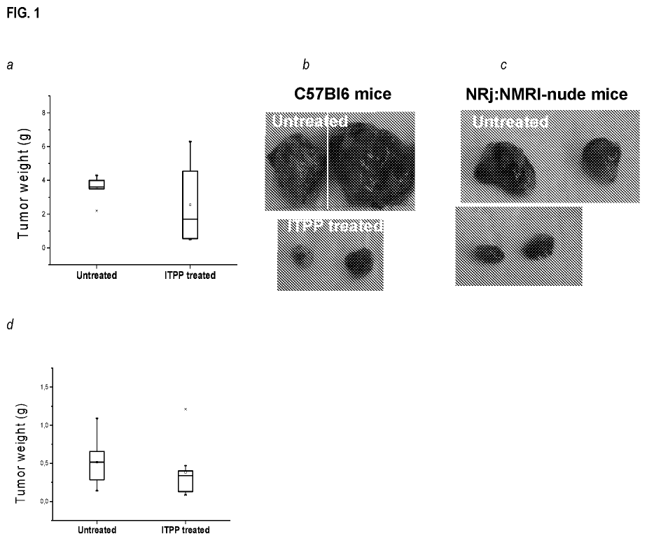

Tumor-bearing animals were treated by ITPP under conditions that normalize vessels²⁵ and a reduction of the tumor size was observed (FIG. 1) on both normal and nude mice for B16F10 melanoma cells (FIG. 1, panels a, b ,c) as well as on 4T1 mammary carcinoma (FIG. 1, panel d). ITPP was shown not to be toxic either for the animals²⁵ or cells treated separately²⁶. Thus the hypothesis of the influence of pO₂ changes on the tumor immune response^{27,28} was tested as we have demonstrated a deep influence on the humoral composition of the tumor microenvironment²⁵.

The selected step of tumor development along treatment, to study the immune reaction was chosen when ITPP-treated tumors were half size of the controls.

This was reached after 23 days for an injection of 104 B16F10 cells both in 057B16 and Rj:NMRI-nu nude mice while it was reached after 31 days for 10⁵ injected 4T1 cells in BALb/c-by mice. The treatments were stopped on day 21. While 50% of tumor growth was observed at day 23 for melanoma cells, the mammary carcinoma reduction was observed up to 50% after arrest of treatment at day 31.

### Effects of ITPP Treatment on the Tumor Microenvironment on NK Cell Response to Tumor

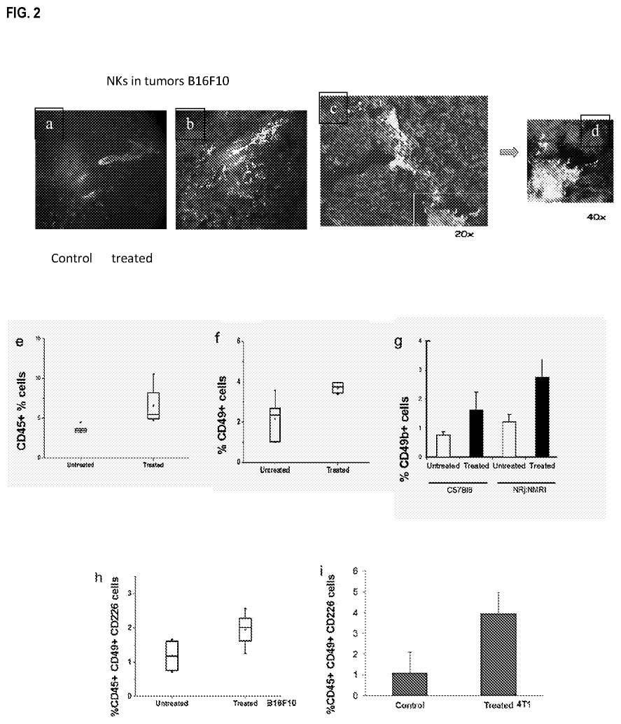

A first approach to analyze the immune cell infiltrate was attempted by immunocytochemical labelling of tumors extracted from animals at day 23 after tumor cells implantation. The infiltrate of NK cells was evidenced by labeling by anti CD49b and endothelial cells revealed by anti-CD31 antibodies (FIG. 2, panels a, b). NK cells were stuck in the vessels of the non-treated tumors (FIG. 2, panel a) while they infiltrated the tumor mass after ITPP treatment (FIG. 2, panel b). This is confirmed on FIG. 2, panel c which displays the distribution of CD49b+ NK cells among the B16F10 transfected by the Luciferase gene and revealed by an anti-luciferase antibody. Inset in FIG. 2, panel c and FIG. 2, panel d indicate the co localization of NK cells with tumor foci.

This ITPP effect on NK cell recruitment could be quantified by immunocytochemical labelling of the tumor cells and tumor stromal cells of the whole microenvironment and assessed by flow cytometry. FIG. 2, panel e shows that the proportion of immune cells (CD45+) in the tumor is higher in ITPP-treated than in control mice. The number of NK cells was doubled in the tumor site (FIG. 2, panel f). The commitment of NK cells in the hypoxia regulation-induced response was confirmed by the reaction observed in nude mice upon B16F10-Luc implantation and treatment by ITPP. In these immune-deficient mice, the T cell immune response is compromised while the NK cells response is increased²⁹. FIG. 2, panel g demonstrates this effect showing the higher proportions of CD49b positive cells (1.5 fold) found in the tumors raised in nude mice compared to normal B57Bl6 mice. This recruitment is similarly enhanced in both types of mice as for the proportions of NK cells found in the tumor before and after ITPP treatments (R=2.25).

Moreover, this increase in intratumor NK cells upon ITPP treatment corresponds to an increase of the numbers of activated (CD45+CD49b+CD226+) NK cells estimated by flow cytometry after ITPP treatment (FIG. 2, panel h) in the melanoma bearing mice.

This effect is confirmed in the mammary carcinoma model with 4T1 cells bearing mice in which ITPP treatment induced a higher recruitment of activated NK cells (FIG. 2, panel i).

### Intra Tumor Evolution of Myeloid Derived Suppressor Cells and Infiltrated Macrophages Phenotype Upon ITPP Treatment

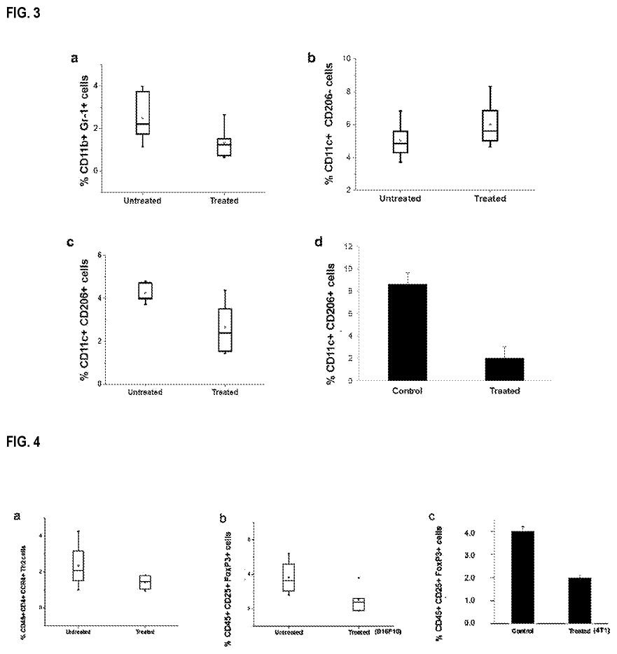

The tumor microenvironment is characterized by the presence of immune suppressor cells (MDSCs) through bone marrow mobilization. These cells help tumor development and escape. In tumors MDSCs express CD11b and Gr-130. ITPP treatment reduced the MDSCs proportion in the tumor (FIG. 3, panel a).

This is accompanied by a tendency to increase the numbers of macrophages expressing the M1 (CD45+CD11c+CD206−) phenotype (FIG. 3, panel b).

Cooperating to tumor immune suppression the proportion of M2 (CD45+CD11c+CD206+) polarized phenotype of macrophages was reduced upon treatment by ITPP, in the melanoma tumor site (FIG. 3, panel c) as well as in the case of mammary carcinoma (FIG. 3, panel d).

### Evolution of the T Cell Populations Infiltrated in the Tumor Upon ITPP Treatment

Proportion of Th2 cells, reflects the inflammatory state which is known to participate to tumor progression. Th2 cells characterized as CD45+CD4+CCR4+, display a proportion decrease in the tumor site upon ITPP treatment (FIG. 4, panel a). The regulatory T cells which cooperate to the tumor development and growth, respond clearly to ITPP treatment as the proportions of CD45+CD4+CD25+FoxP3+ Treg cells were very significantly decreased in B16F10Luc tumors when treated by ITPP (FIG. 4, panel b). This Treg cell numbers reducing effect of ITPP treatment was confirmed in the 4T1 mammary carcinoma bearing BalbC mice (FIG. 4, panel c).

### Tumor Microenvironment Expression of Immune Checkpoint Molecules, Modulation Upon ITPP Treatment

Effect on reducing the proportion of immunosuppressive cell populations as MDSCs, macrophages and Tregs prompted the analysis of the expression of key immune checkpoint molecules as PD-1 and its ligands PD-L1 and PD-L2.

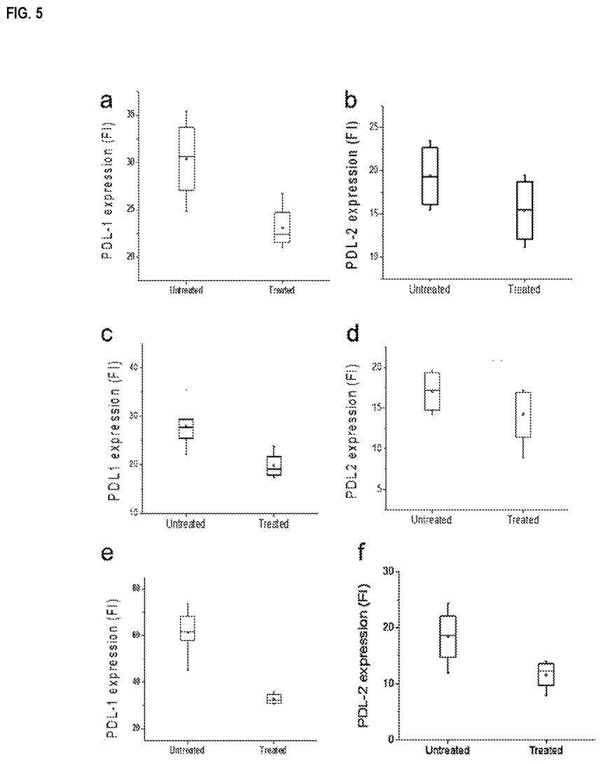

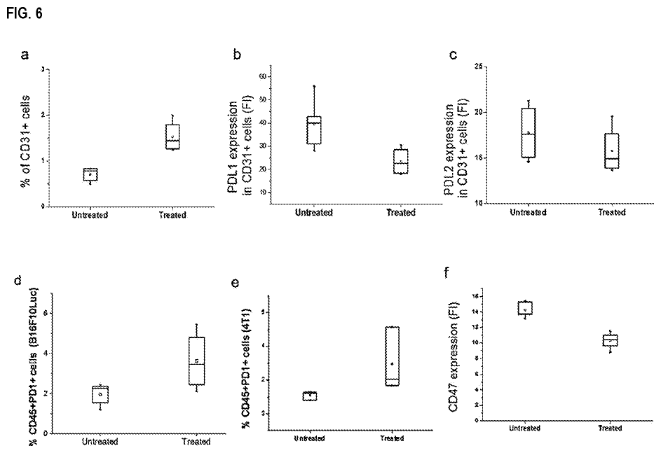

FIG. 5 displays the flow cytometry analysis of cells in the tumor (FIG. 5, panels a, b) and among distinct immune and non-immune cell populations CD45+ (FIG. 5, panels c, d) and CD45− (FIG. 5, panels e, f) respectively. ITPP treatment induced a clear reduction of the expression of PD-L1 and to a lesser extent of PD-L2, on the whole tumor cell populations (FIG. 5, panels a, b) as well as on the separated CD45− cells (FIG. 5, panels c, d) and most clearly on the immune CD45+ cell population (FIG. 5, panels e, f). As shown the PD-1 ligands reduction of expression is strongly reduced in CD45+ cells but also on CD45− cells. This might be attributed to the strong effect shown on the CD31+cells. Endothelial cells proportions are increased and express the junction molecule CD31, which is increased in normoxia as opposed to hypoxia³¹ thus being a criterion of functional vessels²⁵.

FIG. 6 shows that when ITPP is used to treat B16F10Luc melanoma bearing mice the proportion of CD31+ endothelial cells is higher than in non-treated tumors (FIG. 6, panel a). CD31 is more expressed on endothelial cells in normoxia than in hypoxia and is a junction molecule which strengthens the vessels and reduces their permeability^{31,32}. CD31 expression is indicative of vessels normalization. Among CD31+ endothelial cells PD-L1 was evidenced as strongly expressed before ITPP treatment and considerably reduced in treated tumor CD31+ endothelial cells (FIG. 6, panel b). The second PD-1 ligand PD-L2, was also expressed, although to a lesser extent than PD-L1, and was reduced by hypoxia alleviation/vessel normalization resulting from treatment by ITPP (FIG. 6, panel c).

Upon reduction of the PD-1 ligands the ITPP treatment might be accompanied by an increase of the immunocompetent cells expressing PD-1. As above described, CD45+ cell proportions were themselves increased by ITPP treatment (FIG. 2, panel 2). Reduction of the PD-1 ligands, especially on endothelial cells, suggests a possible control on the mechanism of NK cells entry into the tumor. FIG. 6, panels d, e indicates an increase of the number of CD45+ cells in the tumors which were treated by ITPP. As shown on FIG. 2, panels f-i the proportion of NK cells inside the tumor increased upon treatment. This effect concerned more specifically activated NK cells (CD49b+CD226+)³³ and NK cells were able to invade the tumor while they stayed in the vessels in the non-treated tumors (FIG. 2, panel a-d). Indeed, non-treated endothelial cells express PD-L1 and PD-L2 in hypoxia, which is reduced by pO₂ increase as confirmed here upon ITPP treatment (FIG. 6, panel b, c). Moreover, as the Treg cells proportion is considerably reduced they cannot account for the increase of CD45+PD-1+ cells that is reported here among whole tumor populations, both in the case of melanoma (FIG. 6, panel d) as well as mammary carcinoma (FIG. 6, panel e).

The significance of ITPP treatment on the other immune checkpoints expression was assessed on CTLA-4 expression among CD45+ cells. No significant change was observed while the intensity of CD47 expression was found to be reduced on the tumor B16F10 melanoma cells (FIG. 6, panel f). This stem cell marker is a tumor protective molecule again cytotoxic immune cells which promotes evasion of phagocytosis and maintains cancer stem cells. CD47 expression is HIF-1 regulated³⁴ confirming the role of the vessel normalization effect of ITPP treatments and corroborating the stem cell reduction effect in tumors due to ITPP treatments and shown previously²⁵.

### Modulation of the Tumor Microenvironment: Chemokine Receptors Expression Response to Vessel Normalization by ITPP

The intercellular interactions mediated by immune and non-immune stromal cells in the tumor site occur through the fundamental cross talk insured by chemokines and their receptors which rule the metastatic process as largely demonstrated for CXCL12 and its receptor CXCR4 and CCL21 and its receptor CCR7.

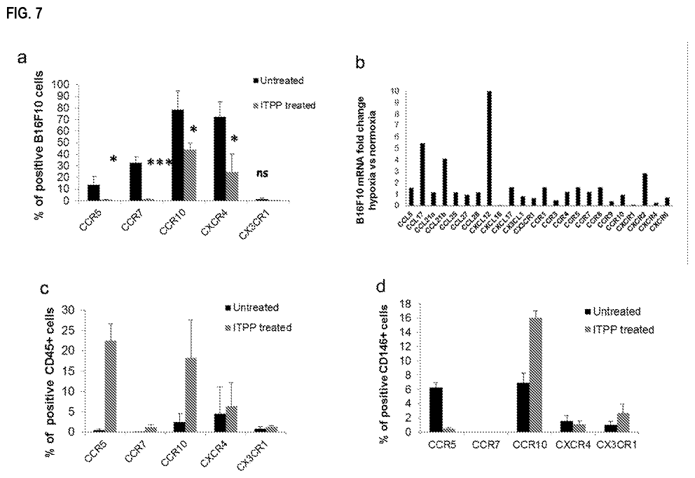

FIG. 7 demonstrates the deep effect that ITPP treatment exerted on the chemokine receptors expression on the tumor cell population but also on the immune cells and the endothelial cells in the tumor site.

A radical effect of ITPP treatment on CCR5 expression was observed on tumor cells (FIG. 7, panel a) corroborating the inverse effect on the CD45+ population and the M1 macrophage repolarization effect³⁵.

The CCR7/CCL21 axis is responsible for melanoma cell metastases into lymph nodes^{36,37,38} and promotes tumorigenesis through stem cells³⁹. A total reduction of the CCR7 expression was observed on tumor cells (FIG. 7, panel a) and no significant expression of this receptor was detected in the other two populations (FIG. 7, panel c, d).

B16F10 cells expressing CCR10, the receptor for CCL27 (cutaneous T-cell attracting chemokine), are reduced upon ITPP treatment while the immune cells expressing it are increased (R=10) (FIG. 7, panel c) as well as the endothelial cells enriched population (R=2) (FIG. 7, panel d); this rules the recruitment of immunocompetent cells⁴⁰.

Tumor cells expressing the receptor for CXCL12 (CXCR4), involved in the metastatic process⁴¹, are strongly reduced (FIG. 7, panel a) which reflects the repair of hypoxic state of tumor cells⁴² and this expression is not affected in CD45+ (FIG. 7, panel c) or endothelial enriched cell population (FIG. 7, panel d).

Moreover, the endothelial enriched cell population selectively displays an induced expression of the fractalkine receptor CX3CR1 upon ITPP treatment (FIG. 7, panel d) which corresponds to a reoxygenation effect and vessels restoration^{43.}

The compared expression of the tumor cells mRNA for chemokines and chemokine receptors (FIG. 7, panel b) indicate a strong relationship between the hypoxic conditions and the humoral microenvironment mainly for some chemokines as CCL17, the activity of which has been shown in the promotion of cancer and its dependence on hypoxia⁴⁴. The main changes appear in the induction of CCL12 and CCL21b mRNAs. This confirms the hypoxia role in the metastatic process of melanoma cells.

### Materials and Methods

### Mice and Tumor Models

C57BL6 mice, BALB/c mice and Nude mice were from Janvier Laboratory (France). Animal care and experimental procedures, performed in accordance with government and institutional guidelines and regulations.

Mouse melanoma model in C57BL6 and Nude mice:

Murine B16F10 cells were implanted in C57BL6 or on Rj:NMRI-nu nude mice leg subcutaneously by injecting a plug of 10⁵ cells in 100 μl Matrigel (BD Biosciences).

### Mice and Tumor Models

C57BL6 mice, BALB/c mice and Nude mice were from Janvier Laboratory (France). Animal care and experimental procedures, performed in accordance with government and institutional guidelines and regulations, were approved by the Ethics Committee.

Mouse melanoma model in C57BL6 and Nude mice:

Murine B16F10 cells were implanted in C57BL6 or on Rj:NMRI-nu nude mice leg subcutaneously as we described previously by injecting a spheroid plug of 10⁵ cells in 100 μl Matrigel (BD Biosciences).

Mouse carcinoma model:

4T1murine mammary carcinoma (10⁴ cells as spheroid plug in Matrigel) cells were injected in the mammary fat pad of BALBc/by mice.

### ITPP Treatment

ITPP was injected intraperitoneally (1.5 g/kg: in saline) twice a week over 3 weeks. It was started on day7 and repeated on day 8 post tumor inoculation (day 0). The following serial treatments were applied on days 15 and 16, 21 and 22. Tumors were extracted and weighted at the indicated times.

### Preparation of Single-Cell Suspensions

Tumor samples were immediately transferred in to PBS on ice. Biopsies were cut into small pieces and filtered through cell strainer after being dissociated by collagenase/dispase (Gibco). The samples were depleted of erythrocytes by red blood lysis buffer (eBiosciences).

In specified experiments, tumors were depleted from CD45+ and/or CD31+ cells by magnetic separation (Easy Sep magnet, StemCell Technologies Inc).

### Cell Staining for Flow Cytometry

Single cell suspensions were stained with mAbs for 1 h at 4° C. Acquisition was performed by one to four color flow cytometry using FACS LSR (Becton Dickinson). Dead cells were excluded on scatter profile. Data were acquired using CellQuest software (Becton Dickinson) on at least 100,000 events. Data were expressed as dot plot or histogram when comparison of fluorescence intensity is needed.

The following directly conjugated rat anti-mouse mAbs were used for the FACS staining: CD45-PerCP, CD11b-APC, CCR4-PE, CCR5-PE, CCR7-PE, CCR1O-PE and CXCR4-PE were from BD Biosciences. CD49b-APC was from Biolegend. CD226-PE, CD4-FITC, CD25-APC, CD8a-FITC, PD-1-PE, CTLA-4-PE, PDL1-PE-Cy7, PDL2-FITC and CD47-APC were from eBiosciences. CD11c-FITC and GR1-FITC were from Milteny. CD206 was from Santa Cruz. We used anti-mouse Foxp3 staining set PE (eBioscience) for Foxp3 staining.

### Immunohistochemistry

Tumor tissues were embedded in tissue freezing medium (Tissue-Tek; Sakura) and snap frozen in liquid nitrogen. Tumor cryosections were fixed and stained with mouse anti-CD31 (rat monoclonal IgG2a; eBiosciences), anti-CD49b (Rat IgM; BD Pharmingen) or anti-Firefly Luciferase (Rabbit IgG; Abcam) before tetramethyl rhodamine isothiocyanate or fluorescein isothiocyanate secondary antibodies were added. Nuclei were stained with bisbenzimide H 33258 (Sigma-Aldrich).

### Quantitative PCR

Extraction of cellular mRNA was performed using the RNeasy Plus mini kit (Qiagen) according to the manufacturer's instructions. Extracted mRNA was eluted in RNase-free water. Absorption spectra were measured on an ND-1000 spectrophotometer (NanoDrop Technologies, Wilmington, DE) before storage at −80° C. RNAs were reverse-transcribed to cDNA using “Maxima First Strand cDNA Synthesis kit for RT-qPCR” (Fermentas). 3 μg of RNA was used for each sample. The obtained cDNA were stored at −20° C. before qPCR. The real-time PCR was performed on LightCycler 480 (Roche) using the “SYBR Premix Ex Taq (Perfect Real Time)” (Takara) and “QuantiTect Primer Assay” (Qiagen) in white 96-well optical microtiter plate (Roche). 2 μL of cDNA were used in a final volume of 20 μL by well. All reactions were completed in triplicate and reported as the average values. For reference, 7 housekeeping genes were tested. Mean and standard deviation were calculated and the gene which had the lowest standard deviation was chosen for reference. For each target gene, mean and standard deviation were calculated then normalized by the corresponding value for reference gene (PPIA) to obtain the ΔCp.

### REFERENCES

1 Hashimoto, T. & Shibasaki, F. Hypoxia-inducible factor as an angiogenic master switch. Frontiers in pediatrics 3, 33, doi:10.3389/fped.2015.00033 (2015).

2 Jain, R. K. & Carmeliet, P. SnapShot: Tumor angiogenesis. Cell 149, 1408-1408 e1401, doi:10.1016/j.ce11.2012.05.025 (2012).

3 Goel, S. et al. Normalization of the vasculature for treatment of cancer and other diseases. Physiological reviews 91, 1071-1121, doi:10.1152/physrev.00038.2010 (2011).

4 Nishida, N., Yano, H., Nishida, T., Kamura, T. & Kojiro, M. Angiogenesis in cancer. Vascular health and risk management 2, 213-219 (2006).

5 Semenza, G. L. Intratumoral hypoxia, radiation resistance, and HIF-1. Cancer Cell 5, 405-406, doi:S1535610804001187 [pii] (2004).

6 Semenza, G. L. Hydroxylation of HIF-1: oxygen sensing at the molecular level. Physiology (Bethesda) 19, 176-182, doi:10.1152/physio1.00001.2004 (2004).

7 Jain, R. K. et al. Biomarkers of response and resistance to antiangiogenic therapy. Nat Rev Clin Oncol 6, 327-338, doi:nrclinonc.2009.63 [pii]10.1038/nrclinonc.2009.63 (2009).

8 Neri, S. et al. Podoplanin-expressing cancer-associated fibroblasts lead and enhance the local invasion of cancer cells in lung adenocarcinoma. International journal of cancer. Journal international du cancer 137, 784-796, doi:10.1002/ijc.29464 (2015).

9 Gil, M. et al. CXCL12/CXCR4 blockade by oncolytic virotherapy inhibits ovarian cancer growth by decreasing immunosuppression and targeting cancer-initiating cells. Journal of immunology 193, 5327-5337, doi:10.4049/jimmuno1.1400201 (2014).

10 Tutunea-Fatan, E., Majumder, M., Xin, X. & Lala, P. K. The role of CCL21/CCR7 chemokine axis in breast cancer-induced lymphangiogenesis. Molecular cancer 14, 35, doi:10.1186/s12943-015-0306-4 (2015).

11 Legler, D. F., Uetz-von Allmen, E. & Hauser, M. A. CCR7: roles in cancer cell dissemination, migration and metastasis formation. The international journal of biochemistry & cell biology 54, 78-82, doi:10.1016/j.bioce1.2014.07.002 (2014).

12 Sceneay, J. et al. Primary tumor hypoxia recruits CD11b+/Ly6Cmed/Ly6G+immune suppressor cells and compromises NK cell cytotoxicity in the premetastatic niche. Cancer research 72, 3906-3911, doi:10.1158/0008-5472.CAN-11-3873 (2012).

13 Hasmim, M. et al. Hypoxia-dependent inhibition of tumor cell susceptibility to CTL-mediated lysis involves NANOG induction in target cells. Journal of immunology 187, 4031-4039, doi:10.4049/jimmuno1.1101011 (2011).

14 Tripathi, C. et al. Macrophages are recruited to hypoxic tumor areas and acquire a pro-angiogenic M2-polarized phenotype via hypoxic cancer cell derived cytokines Oncostatin M and Eotaxin. Oncotarget 5, 5350-5368 (2014).

15 Edin, S. et al. The distribution of macrophages with a M1 or M2 phenotype in relation to prognosis and the molecular characteristics of colorectal cancer. PloS one 7, e47045, doi:10.1371/journal.pone.0047045 (2012).

16 Rodig, N. et al. Endothelial expression of PD-L1 and PD-L2 down-regulates CD8+T cell activation and cytolysis. European journal of immunology 33, 3117-3126, doi:10.1002/eji.200324270 (2003).

17 Noman, M. Z. et al. PD-L1 is a novel direct target of HIF-lalpha, and its blockade under hypoxia enhanced MDSC-mediated T cell activation. The Journal of experimental medicine 211, 781-790, doi:10.1084/jem.20131916 (2014).

18 Yang, H. et al. Expression of PD-L1, PD-L2, PD-1 and CTLA-4 in myelodysplastic syndromes is enhanced by treatment with hypomethylating agents. Leukemia 28, 1280-1288, doi:10.1038/Ieu.2013.355 (2014).

19 Paez-Ribes, M. et al. Antiangiogenic therapy elicits malignant progression of tumors to increased local invasion and distant metastasis. Cancer Cell 15, 220-231, doi:S1535-6108(09)00034-8 10.1016/j.ccr.2009.01.027 (2009).

20 Collet, G. et al. Hypoxia-shaped vascular niche for cancer stem cells. Contemporary oncology 19, A39-43, doi:10.5114/wo.2014.47130 (2015).

21 Jain, R. K. A new target for tumor therapy. The New England journal of medicine 360, 2669-2671, doi:10.1056/NEJMcibr0902054 (2009).

22 Semenza, G. L. Vasculogenesis, angiogenesis, and arteriogenesis: mechanisms of blood vessel formation and remodeling. Journal of cellular biochemistry 102, 840-847, doi:10.1002/jcb.21523 (2007).

23 Zhang, Q. et al. Time-course imaging of therapeutic functional tumor vascular normalization by antiangiogenic agents. Molecular cancer therapeutics 10, 1173-1184, doi:10.1158/1535-7163.MCT-11-0008 (2011).

24 Duarte, C. D., Greferath, R., Nicolau, C. & Lehn, J. M. myo-lnositol trispyrophosphate: a novel allosteric effector of hemoglobin with high permeation selectivity across the red blood cell plasma membrane. Chembiochem: a European journal of chemical biology 11, 2543-2548, doi:10.1002/cbic.201000499 (2010).

25 Kieda, C. et al. Stable tumor vessel normalization with pO(2) increase and endothelial PTEN activation by inositol trispyrophosphate brings novel tumor treatment. Journal of molecular medicine 91, 883-899, doi:10.1007/s00109-013-0992-6 (2013).

26 Kieda, C. et al. Suppression of hypoxia-induced HIF-lalpha and of angiogenesis in endothelial cells by myo-inositol trispyrophosphate-treated erythrocytes. Proceedings of the National Academy of Sciences of the United States of America 103, 15576-15581, doi:10.1073/pnas.0607109103 (2006).

27 Jain, R. K. Antiangiogenesis strategies revisited: from starving tumors to alleviating hypoxia. Cancer cell 26, 605-622, doi:10.1016/j.cce11.2014.10.006 (2014).

28 Goel, S., Wong, A. H. & Jain, R. K. Vascular normalization as a therapeutic strategy for malignant and nonmalignant disease. Cold Spring Harbor perspectives in medicine 2, a006486, doi:10.1101/cshperspect.a006486 (2012).

29 Budzynski, W. & Radzikowski, C. Cytotoxic cells in immunodeficient athymic mice. Immunopharmacology and immunotoxicology 16, 319-346, doi:10.3109/08923979409007097 (1994).

30 Peranzoni, E. et al. Myeloid-derived suppressor cell heterogeneity and subset definition. Current opinion in immunology 22, 238-244, doi:10.1016/j.coi.2010.01.021 (2010).

31 Carreau, A., Kieda, C. & Grillon, C. Nitric oxide modulates the expression of endothelial cell adhesion molecules involved in angiogenesis and leukocyte recruitment. Experimental cell research 317, 29-41, doi:10.1016/j.yexcr.2010.08.011 (2011).

32 Carreau, A., El Hafny-Rahbi, B., Matejuk, A., Grillon, C. & Kieda, C. Why is the partial oxygen pressure of human tissues a crucial parameter? Small molecules and hypoxia. Journal of cellular and molecular medicine 15, 1239-1253, doi:10.1111/j.1582-4934.2011.01258.x (2011).

33 Tahara-Hanaoka, S. et al. Functional characterization of DNAM-1 (CD226) interaction with its ligands PVR (CD155) and nectin-2 (PRR-2/CD112). International immunology 16, 533-538 (2004).

34 Zhang, H. et al. HIF-1 regulates CD47 expression in breast cancer cells to promote evasion of phagocytosis and maintenance of cancer stem cells. Proceedings of the National Academy of Sciences of the United States of America 112, E6215-6223, doi:10.1073/pnas.1520032112 (2015).

35 Halama, N. et al. Tumoral Immune Cell Exploitation in Colorectal Cancer Metastases Can Be Targeted

Effectively by Anti-CCRS Therapy in Cancer Patients. Cancer cell 29, 587-601, doi:10.1016/j.cce11.2016.03.005 (2016).

36 Shuyi, Y. et al. A critical role of CCR7 in invasiveness and metastasis of SW620 colon cancer cell in vitro and in vivo. Cancer Biology & Therapy 7, 1037-1043, doi:10.4161/cbt.7.7.6065 (2014).

37 Gunther, K. et al. Prediction of lymph node metastasis in colorectal carcinoma by expression of chemokine receptor CCR7. International journal of cancer. Journal international du cancer 116, 726-733, doi:10.1002/ijc.21123 (2005).

38 Chouaib, S. et al. Endothelial cells as key determinants of the tumor microenvironment: interaction with tumor cells, extracellular matrix and immune killer cells. Critical reviews in immunology 30, 529-545 (2010).

39 Boyle, S. T. et al. The chemokine receptor CCR7 promotes mammary tumorigenesis through amplification of stem-like cells. Oncogene, doi:10.1038/onc.2015.66 (2015).

40 Pivarcsi, A. et al. Tumor immune escape by the loss of homeostatic chemokine expression. Proceedings of the National Academy of Sciences of the United States of America 104, 19055-19060, doi:10.1073/pnas.0705673104 (2007).

41 Kim, M. et al. CXCR4 signaling regulates metastasis of chemoresistant melanoma cells by a lymphatic metastatic niche. Cancer research 70, 10411-10421, doi:10.1158/0008-5472.CAN-10-2591 (2010).

42 Jin, F., Brockmeier, U., Otterbach, F. & Metzen, E. New insight into the SDF-1/CXCR4 axis in a breast carcinoma model: hypoxia-induced endothelial SDF-1 and tumor cell CXCR4 are required for tumor cell intravasation. Molecular cancer research: MCR 10, 1021-1031, doi:10.1158/1541-7786.MCR-11-0498 (2012).

43 Ryu, J. et al. Activation of fractalkine/CX3CR1 by vascular endothelial cells induces angiogenesis through VEGF-A/KDR and reverses hindlimb ischaemia. Cardiovascular research 78, 333-340, doi:10.1093/cvdcvm067 (2008).

44 Liu, L. B. et al. Chemokine CCL17 induced by hypoxia promotes the proliferation of cervical cancer cell. American journal of cancer research 5, 3072-3084 (2015).

45 Kubota, Y. Tumor angiogenesis and anti-angiogenic therapy. The Keio journal of medicine 61, 47-56 (2012).

46 Duda, D. G. et al. Plasma soluble VEGFR-1 is a potential dual biomarker of response and toxicity for bevacizumab with chemoradiation in locally advanced rectal cancer. The oncologist 15, 577-583, doi:10.1634/theoncologist.2010-0029 (2010).

47 Zhao, Y. et al. Cancer stem cells and angiogenesis. The International journal of developmental biology 55, 477-482, doi:10.1387/ijdb.103225yz (2011).

48 Goel, S., Fukumura, D. & Jain, R. K. Normalization of the tumor vasculature through oncogenic inhibition: an emerging paradigm in tumor biology. Proceedings of the National Academy of Sciences of the United States of America 109, E1214, doi:10.1073/pnas.1203794109 (2012).

49 Tolaney, S. M. et al. Role of vascular density and normalization in response to neoadjuvant bevacizumab and chemotherapy in breast cancer patients. Proceedings of the National Academy of Sciences of the United States of America 112, 14325-14330, doi:10.1073/pnas.1518808112 (2015).

50 Collet, G. et al. Hypoxia-regulated overexpression of soluble VEGFR2 controls angiogenesis and inhibits tumor growth. Molecular cancer therapeutics 13, 165-178, doi:10.1158/1535-7163.MCT-13-0637 (2014).

51 Hong, C. H., Lee, C. H., Chen, G. S., Chang, K. L. & Yu, H. S. STAT3-dependent VEGF production from keratinocytes abrogates dendritic cell activation and migration by arsenic: a plausible regional mechanism of immunosuppression in arsenical cancers. Chemico-biological interactions 227, 96-103, doi:10.1016/j.cbi.2014.12.030 (2015).

52 Kaur, S. et al. CD47 signaling regulates the immunosuppressive activity of VEGF in T cells. Journal of immunology 193, 3914-3924, doi:10.4049/jimmuno1.1303116 (2014).

53 Collet, G. et al. Endothelial precursor cell-based therapy to target the pathologic angiogenesis and compensate tumor hypoxia. Cancer letters 370, 345-357, doi:10.1016/j.canlet.2015.11.008 (2016).

54 Ruf, M., Moch, H. & Schraml, P. PD-L1 expression is regulated by hypoxia inducible factor in clear cell renal cell carcinoma. International journal of cancer. Journal international du cancer 139, 396-403, doi:10.1002/ijc.30077 (2016).

55 Takekoshi, T., Ziarek, J. J., Volkman, B. F. & Hwang, S. T. A locked, dimeric CXCL12 variant effectively inhibits pulmonary metastasis of CXCR4-expressing melanoma cells due to enhanced serum stability. Molecular cancer therapeutics 11, 2516-2525, doi: 10.1158/1535-7163. MCT-12-0494 (2012).

56 Tutunea-Fatan, E., Majumder, M., Xin, X. & Lala, P. K. The role of CCL21/CCR7 chemokine axis in breast cancer-induced lymphangiogenesis. Molecular cancer 14, 35, doi:10.1186/s12943-015-0306-4 (2015).

57 Shields, J. D., Kourtis, I. C., Tomei, A. A., Roberts, J. M. & Swartz, M. A. Induction of lymphoidlike stroma and immune escape by tumors that express the chemokine CCL21. Science 328, 749-752, doi:10.1126/science.1185837 (2010).

58 Benson, D. M., Jr. et al. The PD-1/PD-L1 axis modulates the natural killer cell versus multiple myeloma effect: a therapeutic target for CT-011, a novel monoclonal anti-PD-1 antibody. Blood 116, 2286-2294, doi:10.1182/blood-2010-02-271874 (2010).

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A method for treating cancer, comprising administering an effective amount of an inositol-based agent and an effective amount of one or more immune-modulating agents to a subject in need thereof, wherein the administration is simultaneous or sequential.

2. A method for treating cancer, comprising administering an effective amount of an inositol-based agent to a subject in need thereof, wherein the subject is undergoing therapy with one or more immune-modulating agents.

3. The method of claim 1, wherein the inositol-based agent is myo-inositol tris pyrophosphate (ITPP).

4. The method of claim 2, wherein the inositol-based agent is myo-inositol tris pyrophosphate (ITPP).

5. The method of claim 3, wherein the immune-modulating agent is an immune checkpoint inhibitor (CPI) and/or an immune checkpoint activator (CPA).

6. The method of claim 4, wherein the immune-modulating agent is an immune checkpoint inhibitor (CPI) and/or an immune checkpoint activator (CPA).

7. The method of any one of claim 1, wherein the immune-modulating agent is an agent targeting one or more of a T-cell co-stimulatory or co-inhibitory molecule, an NK cell co-stimulatory or co-inhibitory molecule, a member of the B7 family, a member of the TNF receptor or TNF ligand superfamily, a member of the TIM family, and a member of the Galectin family.

8. The method of claim 1, wherein the immune-modulating agent is an agent targeting one or more of PD-1, PD-L1, PD-L2, CD137 (4-1BB), CD137 ligand (4-1BB ligand), CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4.

9. The method of any one of claim 1, wherein the immune-modulating agent blocks, reduces and/or inhibits the binding of one or more of PD-1, PD-L1, PD-L2, 4-1BB, 4-1BB ligand, CTLA-4, OX-40, OX-40 ligand, HVEM, GITR, GITR ligand, CD27, CD28, CD30, CD30 ligand, CD40, CD40 ligand, LIGHT (CD258), CD70, B7-1, B7-2, ICOS, ICOS ligand, TIM-1, TIM-3, TIM-4, galectin-1, galectin-9, CEACAM-1, CEACAM-4, CEACAM-5, LAG-3, B7-H1, B7-H2, B7-H3, B7-H4, B7-H5, B7-H6, HHLA2, HMGB1, BTLA, CRTAM, CD200, CCR4, and CXCR4 with its binding partner(s).

10. The method of claim 9, wherein the immune-modulating agent blocks, reduces and/or inhibits the activity of PD-1, PD-L1 and/or PD-L2, and/or the binding of PD-1 with PD-L1 and/or PD-L2.

11.-12. (canceled)

13. The method of claim 9, wherein the immune-modulating agent increases, stimulates, and/or enhances the activity of CD137, and/or the binding of CD137 (4-1BB) with CD137 ligand (4-1BB ligand) and/or TRAF2.

14. (canceled)

15. The method of claim 1, wherein the immune-modulating agent is one or more of nivolumab, pembrolizumab, pidilizumab, MK-3475, BMS 936559 MPDL328OA, urelumab, ipilimumab, atezolizumab and avelumab.

16. The method of claim 15, wherein the immune-modulating agent is one of embrolizumab, nivolumab, cemiplimab, atezolizumab, avelumab, and durvalumab.

17.-18. (canceled)

19. The method of claim 1, wherein the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

20.-21. (canceled)

22. The method of claim 1, wherein the subject is refractory to one or more immune-modulating agents.

23. The method of claim 1, wherein the effective amount of the immune-modulating agent is less than an effective amount used in monotherapy for the same cancer and/or a combination therapy with an agent besides an inositol-based agent for the same cancer or wherein the effective amount of the inositol-based agent is less than an effective amount used in monotherapy for the same cancer and/or a combination therapy with an agent besides an immune-modulating agent for the same cancer.

24.-27. (canceled)

28. A pharmaceutical composition comprising an effective amount of an inositol-based agent and an effective amount of one or more immune-modulating agents.

29. The pharmaceutical composition of claim 28, wherein the inositol-based agent is myo-inositol tris pyrophosphate (ITPP).

30. The pharmaceutical composition of claim 29, wherein the immune-modulating agent is an immune checkpoint inhibitor (CPI) and/or an immune checkpoint activator (CPA).

31.-36. (canceled)

37. The pharmaceutical composition of claim 30, wherein the immune-modulating agent is one or more of nivolumab, pembrolizumab, pidilizumab, MK-3475, BMS 936559, MPDL328OA, urelumab, ipilimumab, atezolizumab and avelumab.

38.-41. (canceled)
